SEQUENCE LISTING (41 sequences)

SEQ ID NO: 1 - DNA, 3017 bp, Aurantiochytrium sp.
  misc_feature: TEF promoter
agcaggagtggattcggaaggccccaaatggatggcacgagcgagctccttccttcctctcgcgccgcactctctccctccctccctcctctctctcgcgcgcgagtctcgctcactctcctttgcaagagcaacaagcagcctcggcagcgaatgaatgagagtcctccttcgcttctttctcgattcaactcgaagaatgaatgattttcattgctcaaataaataaataaataaataaataaataaataattattgttccattcatggattggcaattacttggttagctagctagctagctagtgagtgagttagtgggttttagtagtgctaacggatggcggcaaagacctcgtcaaaaaaaaatcaagaaagcaagatgaagaagggcctgtgattcaagacccgcgttctgtctcgcttactgcgtggagtgcggagctccgacacgcttgaaattggccaaaagctgcacttcgcgccaccctctgcgccccgaaggtggctttgggccggagcaccaagtttagcgcactgtaaaaaggcgcgaaactttgttggagaagccaattaattaattaattaattaattaatcctttcgacgaaaactaaagaagaagaaagaaatcaagtttccgccctataaaatatcccttcttcacacttccttcattttgtagttagatgataggcagcgaaaggactaaaggtgaaaggcgtagggaccacataggcgcgctagggcggagggaaagatacaaatggcctcagaaaggaagaagaagaggcctcgcggaggaaggatgctgaagcaggaaagatacagcgaaagagaaatcctgtatcttccacagtggatggacaccttcgaggcctgcataagtccacatcactcgctattcaatcattgaattggtcatttaattcaagcatttaattcaatcatgtcttcatgcaatccaccgtccaacaacagagcgcatagaagatgttatccaggtaaggctgcaataatacgcagtttgagttttctattttaaaagtaagtttaaaacttaaaaatttcatacttatgcatgctattcaaaataagattgtatcatcctaaagtattcttcttctcgttcttcttctaatcggaacagagacaactttggtgggtttgcgggcctttgagagaaagaaaaaaaactctcaaaagaaaccaggcttccgaggccgacttgcgcagctctggattgaggttccttcgatcgctcgcttcaccttcctggcccgcgcatgcctcgctctgggtacacagctgagtgagtgagcgaaagatgagcgaatgaatgcaatatttttctattttctattcatttaactgtacttaattaattgattattgattgattgattgattgattgattgattgattaatgactctcgcttctgagaatacatctgttctcatcttcatcgtcacgtcagaatggaaggatgagaaatgaaaagaattcgatcactttcccgccttcttgctagctcatgctcctttcccgccaaaaagaaagaagaggaaagcaccccgaagaaaagaaagaaatcacccaaacaccctcctccttcctcgtccacagacagctcagaataatgaaagctatctttccatcgctcttgacctaactctctttctgctcctgtaaattcatccaacaaatgtttagtctcagaaacccatctgcctcatactactacttactaccttccttacttgaaagcaggcaggctcacggccagcttggcagataggatagttctcatatctattgctgatcgttcccgtttctttctcaaagcaaagtcttttctcttcattccttttctttttcttttcttttcaggctctccacgttttcaggagtagtacatttgctacttagtaattagaaagcttagtactttttgcttttctggattctgaagacttggaaatagaaagaaattaaaaatctttttcttctttctttcagcctttgctggactccctcgcacgcctccttcttccccagccatccatcagcgggcactccacccgcgcttcaacgctcgctcgagtgcgtgcttatttgccttcaacgcggcgcggcggttaatatagtcccagcactccttaaggggggcatcgcagggattatctttttaaaacctgtcacggagttacattttccctcgcatcaaagtgttcccggccgcgtcgcacatctaagttttataacctacacccctcgtggggtaggggcgaattctatgtacacagcacctcagaacttgcgcgcgttccgtgacaaatgaggggtgtggcggcgcattcggccgcatcgccacattcagatatctaacatacccccccttcgcgatgagtggcaggcgaggcggattcgctcgcgagaggcgaggtgccacagcagaccagtaacgaggagccaaggtaggtgaccaccgacgactacgaccacgaccacgaccacagccacggcggctgcagccacgggacgcctcgcatggcagcgcatcagcaccagcaacgacagctgcgaggagcgcagggccgatctggacgcgccggagccgcacgaccaatgccgacgcaacgctgattcttctggattacctctacacatgcatatatgtgtagaggtgcggatgaaatgccctgcgaataaatgaatggcttcgagtttgcctgccgtatgctcgaaagtgcgtgtgcagacacaggcacgaccgagaggacaacagtctgtgcttacctcaccagcacattcttgcaacgccatacgaagcacgcgaaatcttgtggctcagagaggaaggcattcgtgtacgggaacgtggggaacgctatcaatttggaattcaaaatgagtgaaccagacaactaactgtgacttgaactgttgctccacgcatcaaaaccaaacccttaacagaagtagaccagttcgaagctactagcaccaaacaaa

SEQ ID NO: 2 - DNA, 81 bp, Batrachochytrium dendrobatidis
  misc_feature: SP579_nucleotide_sequence
atgccctttaaccgcctttctcttccttgccttcttcttgctctcattgctagcctcttcattcatgctgctcaagctggt

SEQ ID NO: 3 - DNA, 642 bp, Homo sapiens
  misc_feature: natalizumab_light_chain_nucleotide_sequence
gacatccaaatgacacaaagcccttctagccttagcgcttctgttggtgatcgtgtgaccattacatgtaagacatctcaggacatcaacaagtacatggcctggtaccagcagacacctggtaaagctcctcgtcttctcatccactacacctctgctcttcaacctggcattccttctcgcttttctggttctggttctggtcgcgattacaccttcaccattagctctcttcagcctgaggacattgccacatactactgccttcagtacgacaacctctggacatttggtcaaggcacaaaggtggagatcaagcgtacagttgctgctcctagcgtgttcatttttcctccttctgatgagcagctcaagtctggtacagcttctgttgtttgcctcctcaacaacttctaccctagagaagctaaggtgcagtggaaggttgataacgctcttcaatctggcaactctcaggagtctgttacagagcaagacagcaaggatagcacctactctctttctagcacccttacccttagcaaggctgattacgagaagcacaaggtttacgcttgcgaggttacacatcagggtctttcttctcctgtgaccaagagctttaaccgtggtgaatgttaa

SEQ ID NO: 4 - DNA, 1353 bp, Homo sapiens
  misc_feature: natalizumab_heavy_chain_nucleotide_sequence
caggttcaactcgttcagtcaggtgctgaggtgaaaaaacctggtgcttctgtgaaggtgagctgtaaagctagcggcttcaacatcaaggacacctacattcactgggttcgccaagctcctggtcaaagattagagtggatgggtcgcattgaccctgctaatggttacaccaagtacgaccccaagtttcaaggtcgcgtgacaattaccgcagacacatctgcttctaccgcctatatggagcttagctctcttcgttctgaggatacagccgtgtactattgtgctcgtgagggttactacggcaactatggtgtttacgccatggactattggggccaaggtacacttgtgactgtgtcttctgctagcacaaagggtcctagcgtgtttcctttagctccttgttctcgctctacctctgagtctacagctgctttaggctgccttgtgaaggactactttcctgaacctgtgaccgtgtcttggaactctggtgctcttacatctggcgttcacacctttcctgctgttcttcagtcttctggcctctactctcttagctctgtggttacagtgcctagctcttctcttggcacaaagacatacacctgcaacgtggatcacaagcctagcaacacaaaggtggataagcgcgttgagagcaagtatggtcctccttgtccttcttgtcctgctcctgagtttcttggtggtccttctgtgttcctcttccctcctaaacctaaggacaccctcatgatttctcgcacacctgaggttacttgcgtggttgttgacgtttctcaggaggatcctgaggtgcagtttaactggtacgttgatggcgttgaggtgcataacgctaagacaaaacctcgtgaggagcagttcaacagcacatatcgcgttgtgagcgtgcttacagtgcttcatcaggattggcttaacggcaaggagtacaagtgcaaggtgagcaataagggccttcctagcagcattgagaagaccattagcaaggctaagggccaacctagagagcctcaagtttacacactccctccttctcaagaggagatgaccaagaaccaggtgtctcttacctgccttgttaagggcttctaccctagcgacattgctgttgaatgggagtctaacggccaacctgagaacaactacaagacaacaccccctgtgcttgactctgatggcagctttttcctctactctcgccttacagtggacaagtctcgttggcaagagggtaacgtgtttagctgctctgtgatgcatgaggctctccataaccactacacccagaagtctcttagcctttctctcggcaagtag

SEQ ID NO: 5 - DNA, 4251 bp, Artificial Sequence
  Synthetic
  misc_feature: NLS-Flag-Cas9 (pAM-001)
atgcccaagaaaaagcggaaggtcggcgactacaaggatgacgatgacaagttggagcctggagagaagccctacaaatgccctgagtgcggaaagagcttcagccaatctggagccttgacccggcatcaacgaacgcatacacgagacaagaagtactccatcgggctggacatcgggacgaactccgtgggatgggccgtgatcacagacgaatacaaggtgccttccaagaagttcaaggtgctggggaacacggacagacactccatcaagaagaacctcatcggggccttgctcttcgactccggagaaaccgccgaagcaacgcgattgaaaagaaccgccagaagacgatacacacgacggaagaaccgcatctgctacctccaggagatcttcagcaacgagatggccaaggtggacgactcgttctttcatcgcctggaggagagcttcctggtggaggaagacaagaaacatgagcgccacccgatcttcgggaacatcgtggacgaagtggcctaccacgagaaataccccacgatctaccacttgcgcaagaaactcgtggactccacggacaaagcggacttgcggttgatctacttggccttggcccacatgatcaaatttcggggccacttcctgatcgagggcgacttgaatcccgacaattccgacgtggacaagctcttcatccagctggtgcagacctacaaccagctcttcgaggagaaccccatcaatgcctccggagtggacgccaaagccatcttgtccgcccgattgtccaaatccagacgcttggagaacttgatcgcacaacttcctggcgagaagaagaacggcctcttcggcaacttgatcgcgctgtcgctgggattgacgcctaacttcaagtccaacttcgacttggccgaggacgccaagttgcaactgtccaaggacacctacgacgacgacctcgacaacctgctggcccaaattggcgaccaatacgcggacttgtttttggcggccaagaacttgagcgacgccatcttgttgagcgacatcttgcgcgtgaatacggagatcaccaaagcccctttgtccgcctctatgatcaagcggtacgacgagcaccaccaagacttgaccctgttgaaagccctcgtgcggcaacaattgcccgagaagtacaaggagatcttcttcgaccagtccaagaacgggtacgccggctacatcgacggaggagcctcccaagaagagttctacaagttcatcaagcccatcctggagaagatggacggcaccgaggagttgctcgtgaagctgaaccgcgaagacttgttgcgaaaacagcggacgttcgacaatggcagcatcccccaccaaatccatttgggagagttgcacgccatcttgcgacggcaagaggacttctacccgttcctgaaggacaaccgcgagaaaatcgagaagatcctgacgttcagaatcccctactacgtgggacccttggcccgaggcaattcccggtttgcatggatgacgcgcaaaagcgaagagacgatcaccccctggaacttcgaagaagtggtcgacaaaggagcatccgcacagagcttcatcgagcgaatgacgaacttcgacaagaacctgcccaacgagaaggtgttgcccaagcattcgctgctgtacgagtacttcacggtgtacaacgagctgaccaaggtgaagtacgtgaccgagggcatgcgcaaacccgcgttcctgtcgggagagcaaaagaaggccattgtggacctgctgttcaagaccaaccggaaggtgaccgtgaaacagctgaaagaggactacttcaagaagatcgagtgcttcgactccgtggagatctccggcgtggaggaccgattcaatgcctccttgggaacctaccatgacctcctgaagatcatcaaggacaaggacttcctggacaacgaggagaacgaggacatcctggaggacatcgtgctgaccctgaccctgttcgaggaccgagagatgatcgaggaacggttgaaaacgtacgcccacttgttcgacgacaaggtgatgaagcagctgaaacgccgccgctacaccggatggggacgattgagccgcaaactgattaatggaattcgcgacaagcaatccggaaagaccatcctggacttcctgaagtccgacgggttcgccaaccgcaacttcatgcagctcatccacgacgactccttgaccttcaaggaggacatccagaaggcccaagtgtccggacaaggagactccttgcacgagcacatcgccaatttggccggatcccccgcaatcaaaaaaggcatcttgcaaaccgtgaaagtggtcgacgaactggtgaaggtgatgggacggcacaagcccgagaacatcgtgatcgaaatggcccgcgagaaccaaaccacccaaaaaggacagaagaactcccgagagcgcatgaagcggatcgaagagggcatcaaggagttgggctcccagatcctgaaggagcatcccgtggagaatacccaattgcaaaacgagaagctctacctctactacctccagaacgggcgggacatgtacgtcgaccaagagctggacatcaaccgcctctccgactacgatgtggatcatattgtgccccagagcttcctcaaggacgacagcatcgacaacaaggtcctgacgcgcagcgacaagaaccggggcaagtctgacaatgtgccttccgaagaagtcgtgaagaagatgaagaactactggcggcagctgctcaacgccaagctcatcacccaacggaagttcgacaacctgaccaaggccgagagaggaggattgtccgagttggacaaagccggcttcattaaacgccaactcgtggagacccgccagatcacgaagcacgtggcccaaatcttggactcccggatgaacacgaaatacgacgagaatgacaagctgatccgcgaggtgaaggtgatcacgctgaagtccaagctggtgagcgacttccggaaggacttccagttctacaaggtgcgggagatcaacaactaccatcacgcccatgacgcctacctgaacgccgtggtcggaaccgccctgatcaagaaataccccaagctggagtccgaattcgtgtacggagattacaaggtctacgacgtgcggaagatgatcgcgaagtccgagcaggagatcggcaaagccaccgccaagtacttcttttactccaacatcatgaacttcttcaagaccgagatcacgctcgccaacggcgagatccgcaagcgccccctgatcgagaccaacggcgagacgggagagattgtgtgggacaaaggaagagattttgccacagtgcgcaaggtgctgtccatgcctcaggtgaacatcgtgaagaagaccgaggtgcaaacaggagggttttccaaagagtccattttgcctaagaggaattccgacaagctcatcgcccgcaagaaggactgggaccccaagaagtacgggggcttcgactcccccacggtggcctactccgtgttggtggtggccaaagtggagaaagggaagagcaagaagctgaaatccgtgaaggagttgctcggaatcacgatcatggaacgatcgtcgttcgagaaaaaccccatcgacttcctcgaagccaaagggtacaaagaggtgaagaaggacctgatcatcaagctgcccaagtactccctgttcgagctggagaacggccgcaagcggatgctggcctccgccggggaactgcagaaagggaacgaattggccttgccctccaaatacgtgaacttcctctacttggcctcccattacgaaaagctcaaaggatcccctgaggacaatgagcagaagcaactcttcgtggaacaacacaagcactacctggacgagatcatcgagcagatcagcgagttctccaagcgcgtgatcctcgccgacgccaacctggacaaggtgctctccgcctacaacaagcaccgcgacaagcctatccgcgagcaagccgagaatatcattcacctgtttaccctgacgaatttgggagcccctgccgcctttaaatactttgacaccaccatcgaccgcaaaagatacacctccaccaaggaagtcttggacgccaccctcatccaccagtccatcacgggcctctacgagacgcgcatcgacctctcccaattgggcggcgactaa

SEQ ID NO: 6 - DNA, 788 bp, Streptococcus pyogenes
  misc_feature: hsp60 promoter
acgttcttcgcgaagtcaatccattccccgcgttccccaaatgagggttcgcggtcgaacccgggggctgagaagggccttaaaagcgcgggtttaaagagggatcgggagcggcgggagacaagggattaaggtggaagtggacccttttccagaagggagaaaagcacgagcgggagattgactggtgcagcagatcccgaacgacgtcttcgacaggtacgtgcctcagattgaggtgccgctcatgcggcactgtattcaagcgctctagctggccgccatgttgctgccactctgtttgccgctcgcggccacacggctgccgccaggaccacccaccacccgctccagctgccgtgagctgagcttacctatggacgcatgagcggctccaagccacacgtcctgtctggtgaatatccaacttgacgtcgcggctttgtctccatcattctagctgcgaatctggattgctgaggagatcatcgcttctgcgcggtgtgacgccggcttcagccgcgatagattgatttggatggaagcgaccaagcagagcgtcgcatctccttaccgggtattagggttctgtagatccaaaagacctagtttatgtattgagtggcagagacgaaaaattggctcaggctaatttgaatggctgtggctaagtccttaaatgcttggtggacaatcgatggaagaagagcaaagtgaacaaaaaagactgacctttcaagtttaatttatttgcaatccacaggcgacaaaacaaaacacaaataaaa

SEQ ID NO: 7 - DNA, 21 bp, Artificial Sequence
  Synthetic
  misc_feature: primer oSGI-JU-1360
cttaaatgcttggtggacaat

SEQ ID NO: 8 - protein, 434 aa, Aurantiochytrium sp.
  misc_feature: mannosyl transferase (alg3) SG4EUKT579099
MSLRASKDALVRLRGALDNASTQWWWWAMAATADLALSLLIVKLVPYTEIDFKAYMQEVEGPLLHDEWDYTKLRGDTGPLVYPAGFVYIYMGIRWLTEDGTNLWRGQILFASLHAILVYLVLGSIYYQPDASKDPRRVPFWVGPLAVLSRRVHSIFVLRLFNDGIAMVFMYAAVYMYVRRRWTLGTAFFSAALSVKMNILLFAPGLAVLMLEATGLASSILQAVICVASQIALALPFLQVNAAGYLNRAFELGRVFTYKWTVNFKFLSPEAFVSKALAQGLLSATLLTWVGFGSRHFASSHTGGLRGLVYTSIVRPLKAPLEDTISTVQMHDWKLHVLTLLFTSNFIGIVFARSIHYQFYTWYFHTVSFLVYASGGNFALSLLICVSLEVCFNVYPSTAESSAILQATHLVLLLRLATRKPCPLTAQSKRPKQA

SEQ ID NO: 9 - protein, 434 aa, Aurantiochytrium sp.; identical to SEQ ID NO: 8
  misc_feature: mannosyl transferase (alg3) SG4EUKT579102

SEQ ID NO: 10 - protein, 434 aa, Aurantiochytrium sp.
  misc_feature: mannosyl transferase (alg3) SG4EUKT561246
MSFRASKDALVRLRGALDNASTQWWWWAMAATADLALSLLIVKLVPYTEIDFKAYMQEVEGPLLHDEWDYTKLRGDTGPLVYPAGFVYIYMGIRWLTEDGTNLWRGQILFASLHAILVYLVLGSIYYQPDASKDPRRVPFWVGPLAVLSRRVHSIFVLRLFNDGIAMVFMYAAVYMYVRRRWTLGTAFFSAALSVKMNILLFAPGLAVLMLEATGLASSILQAVICVASQIALALPFLQVNAAGYLNRAFELGRVFTYKWTVNFKFLSPEAFVSKALAQGLLSATLLTWVGFGSRHFASSHTGGLRGLVYTSIVRPLKAPLEDTISTVQMHDWKLHVLTLLFTSNFIGIVFARSIHYQFYTWYFHTVSFLVYASGGNFALSLLICVSLEVCFNVYPSTAESSAILQATHLVLLLRLATRKPCPLTAQSKRPKQA

SEQ ID NO: 11 - DNA, 1305 bp, Aurantiochytrium sp.
  misc_feature: mannosyl transferase SG4EUKT579099
atgtctttgcgtgcgagtaaggatgccctcgtacgtcttcgaggggccctcgacaatgcaagcactcagtggtggtggtgggccatggcagccacggcagacttggcacttagcctgcttattgtgaaactcgtgccttatacggagatcgactttaaagcgtacatgcaagaggttgaaggccccctattgcatgatgaatgggactatacaaagctcaggggcgacacaggcccgctggtttatcctgccggttttgtgtatatttatatgggcatccgctggctcactgaagacggcacgaacctgtggcgaggccagatactttttgcaagtctgcatgcaattcttgtttaccttgtacttggatccatatattaccagccagatgcatcaaaagatcctcgcagagtgccgttctgggtaggacctctagcagtattatcgagacgtgtgcattcaatctttgttctgaggctcttcaacgacggcattgctatggtgtttatgtatgcagcagtatatatgtatgtgcggaggcgttggacgctaggtacggctttcttcagcgcagcacttagcgtgaaaatgaatatactcctatttgcgccaggattagccgtgttgatgctcgaggctacgggtttggcgtcgagcatactgcaggcagtgatctgcgtagcatcacagattgccttagctttgccgttcctccaagtcaacgcagccgggtatctaaatcgggcttttgagctaggtcgtgtctttacgtacaaatggacagtaaacttcaagtttctcagccctgaagcttttgtgagtaaggcacttgcccaaggcctgctgtctgccactttacttacatgggtcggctttgggtctcgccactttgcttcctctcacacaggtggtcttcgcggccttgtgtacacgagcattgttcgaccactgaaagctccgcttgaagacacaatttcaaccgtccaaatgcatgactggaaacttcacgttttgacgctcctattcacaagcaactttattggcatcgtttttgcgcgaagcatccattaccaattctacacttggtactttcacactgtctcattcttagtgtacgccagtggtggaaacttcgcgttgtctcttcttatttgcgtttctctagaagtatgctttaacgtgtatccttcaacagcagaatcgagtgctatcttgcaggcaactcatcttgttttgttattgagacttgctacacgaaaaccttgcccacttacagcacagagcaagcgccctaaacaagcatga

SEQ ID NO: 12 - DNA, 1305 bp, Aurantiochytrium sp.; identical to SEQ ID NO: 11
  misc_feature: mannosyl transferase (alg3) SG4EUKT579102

SEQ ID NO: 13 - DNA, 1305 bp, Aurantiochytrium sp.
  misc_feature: mannosyl transferase (alg3) SG4EUKT561246
atgtctttccgtgcgagtaaggatgccctcgtacgtcttcgaggggccctcgacaatgcaagcactcagtggtggtggtgggccatggcagccacggcagacttggcacttagcctgcttattgtgaaactcgtgccttatacggagatcgactttaaagcgtacatgcaagaggttgaaggccccctactgcatgatgaatgggactatacaaagctcaggggcgacacaggcccgctggtttatcctgctggttttgtgtatatttatatgggcatccgctggctcactgaagacggcacaaacctgtggcgaggccagatactttttgcaagtctgcatgcaattcttgtttaccttgtacttggatccatatactaccagccagatgcatcaaaagatcctcgcagagtgccgttctgggtaggacctctagcagtattatcgagacgtgtgcattcaatctttgttctgaggctcttcaacgacggcattgctatggtgtttatgtatgcagcagtatatatgtatgtgcggaggcgttggacgctaggtacggctttcttcagcgcagcacttagcgtgaaaatgaatatactcctatttgcgccaggattagccgtgttgatgctcgaggctacgggtttggcgtcgagcatactgcaggcagtgatctgcgtagcatcacagattgccttagctttgccgttcctccaagtcaatgcagcagggtatctaaatcgggcttttgagctaggtcgtgtctttacgtacaagtggacagtaaacttcaagtttctcagccctgaagcttttgtaagtaaggcacttgcccaaggcctgctgtctgccactttacttacatgggtcggctttgggtctcgccattttgcttcctctcacacaggtggccttcgcggccttgtgtacacgagcattgttcgaccactgaaagctccgcttgaagacacaatttcaaccgtccaaatgcatgactggaaacttcacgttttgacgctcctattcacaagcaactttattggcatcgtttttgcgcgaagcatccattaccaattctacacttggtactttcacactgtctcattcttagtgtacgccagtggtggaaacttcgcgttgtctcttcttatttgcgtttctctagaagtatgctttaacgtgtatccttcaacagcagaatcgagtgctatcttgcaggcaactcatcttgttttgttattgagacttgctacacgaaaaccttgcccacttacagcacagagcaagcgccctaaacaagcatga

SEQ ID NO: 14 - DNA, 103 bp, Aurantiochytrium sp.
  misc_feature: alg3_gRNA
gcguacaugcaagagguugaguuuuagagcuagaaauagcaaguuaaaauaaggcuaguccguuaucaacuugaaaaaguggcaccgagucggugcuuuuuuu

SEQ ID NO: 15 - DNA, 120 bp, Artificial Sequence
  Synthetic
  misc_feature: primer oCAB-0341
taatacgactcactatagcgtacatgcaagaggttgagttttagagctagaaatagcaagttaaaataaggctagtccgttatcaacttgaaaaagtggcaccgagtcggtgcttttttt

SEQ ID NO: 16 - DNA, 120 bp, Artificial Sequence
  Synthetic
  misc_feature: primer, oCAB-0342
aaaaaaagcaccgactcggtgccactttttcaagttgataacggactagccttattttaacttgctatttctagctctaaaactcaacctcttgcatgtacgctatagtgagtcgtatta

SEQ ID NO: 17 - protein, 426 aa, Aurantiochytrium sp.
  misc_feature: SFT1, heparin sulfate 2-O-sulfotransferase
MSSAKENNGSTLTQRPAAKKANETKETKVEGEAAVKADNATPTNAPKPRVARKKRDKTMDYLKLAGALLLMAFTMVVNPMTLGHFGYFVQDCEGKCFVTAEEDNRRCGPTDTTRLIYTRIPKTGSTTVYDILHSLNRRKNFNTVKLGEFDLAVQTVDSKTNAGTAGYNDPNIQRATANRFKFLKDNGEAVVFPPYAGRNRTFFEGHVFHLNWPMALLMPPPFWYKLVPEPILEYLHLEKPSKKVMENTIEFSVVRKPQDRLRSMYYYARLHARSETWRQAYREQFGELEFDECVLDPVCAEKNELRRWCSLQTQFLCGYDKECESPSDAMLETALKNLNDNIFIVGTTERLTDFIELMEKLLPTYFEGATELAKVTNSKQYTDTRRELKFSSEVQAVLDDVCELDNKLYAAVDKLFSERFDECMSP

SEQ ID NO: 18 - protein, 524 aa, Aurantiochytrium sp.
  misc_feature: SFT2, sulfotransferase
MPWLVKEEESKEEKLLFIHVPRCGGTSLIKLYNVEAKCRRVSNLYHKIGQLYFFYRYRLLEQSNFPIKSFENLYALTVFITATIVYFAVPDWGIAACSWDRWCPPPISISFWVCGFATFFMSTFVMAANVMGRRDIVRRLCMWAFGKIMFGFGGAPQYLHGVNYDGYLMHYTAEKLISHKFIKPADLQDSFAIVRNPFSRMVSVYRYNQAHSCESFAHFVKEWRKKFTVFEMTGSTEEWDVYCHVLPMHSFTHFRGQQLVPYIIRQEDLKIIKRRDGQETDLYQSRYADLPEKVIYALTNMPHANKRASKGPAWYDLYDQETMDVVLEMFAQDFVTYGYETTMPKRDDLKPKYTLAQLTAKLHPANVNKPAKKNTASAATMKRNKTVPEDFDPERIIVNFDDDDDHCDTGSSVSSLSASSASSSSRPVEDDEEDDDAHDRISSEDSAQLEAHIRPASEEIYVNVRPADASSASASALPPVASAPATTGRRGRVQNESPQPASTSGIKVASSFRDNSDPDVITHV

SEQ ID NO: 19 - protein, 449 aa, Aurantiochytrium sp.
  misc_feature: SFT2, sulfotransferase
MSVVERRLRDEAEAKSLEQQQRLRQCTVFFGFFILFLIIYTSRYADSITEGVGGLPVLLRDSGFTNNSQTSDLDRGDDNSYGTSSDSNEQPEDTTTDKAEAEMLTSKLSSFGLAELGTFFDEDEVKRNRALAEQELVALAAEDAEFDLFKTTGNRNLIFIKLHKTGSTSVAASLLRVATEYKKKVLTSHSCSDSKDYEMYFMHAPRSEWMNQCIQNPVYVTVLREPMSRHVSWNTWRLNRNYFINLPSRQCNYEGANPDVAPLRKPFQCRDDAARKKMTLRRYLKKLELTNPASRPCESCNWLNPSNEVAHPGNPAEEVARAEQVYSMVGITSNLDDFLLLLALRFGWSLESILYEKCKQQGKAGISLRDVKKYPDMYAKLENITTRESAVYNPFEAKFKAYLGRLGPGFARLLENFKRGLNNYHSKIDEAKGENKDRWIPAGKNSFYC

SEQ ID NO: 20 - protein, 510 aa, Aurantiochytrium sp.
  misc_feature: SFT4, sulfotransferase
MPWLVMDEVLFFHVPRCGGTSITKHHRVGYKARQGMNPYFKFGLVYYYYRYWLLEQSNFPIFTWENLLAMTQITIAIFIYFFMTPIPPAPYVMWTMASLTFTMSTFIWTAPITMRNNTLRWLLMLFQSKVLCGFGGETRYMTGTNDKGYLFHFTANRAIKYNYVTPEQVRRCSFSIVRNPYSRMVSMYEYNKRPFESFSHFVRAFHHEYWHTYIGRNTTECKYVYCHVLPMFEYTHKNGEQIVSCVIKQEHLKNLVATDWEGSNVPEPIQRAMTGIPHANKRSRKVAWQRYFTQETMDLTYEMYHKDFEIFGYDIDIPGRPDISVSRSVLESIRQDPSGAMRQGSDVDVCSPNASSGYARVDAGGESSRRVAIEVDYQDDDLSAPVLNGDRQHGEANGAIPTVVVMNDDAEAIKDKGATSTQKENEEDEGEEVESKEIHRATSFKRKGSLDIANNPPLDNTTKNGSNENGSSRSFGRSSLRIMPAPVVSESELAARREASVFVLSPTRQN

SEQ ID NO: 21 - protein, 462 aa, Aurantiochytrium sp.
  misc_feature: SFT5, sulfotransferase
MNVKSTKEHQKSPQARIREILVNYRALIIGAWFGLVIYVIMMLNSSSSSIQNITLLITSNSDPALANSAEAEESFYNEELKDDMLEEGISITPSKTAISKDEPIGARQRDKLAVFLHIHKGGGSTMCYLARLNNEKSFGGNCNAKSRTTRLKLASGSKDVVENTYGNFKSQNLTFVANEWMLVESTPMSDDLVYITMLRNPLARMESHFDMAMDQGLQRLMRNEELYSCRYADGIPSAKELKVRNQDLEVASRARTYFALSTPDNWQTRAVCGPACAQVPFGKLTDEHLDIAKRRLESTFAVVGILEHFVETITLIHDHLQWKMKPEKNFMQHKGTSHGGMSVLERVEKESAKRPELLSFARWFHATNQFDIELYNFARYLFKQQADARNIPLDLGPDVELDFVSPNQKTTKSSKLRKMLSDYSDNKNCTTQCCGKRCGPIGFYWYSTAVSLNMVPRKLGHC

SEQ ID NO: 22 - protein, 739 aa, Aurantiochytrium sp.
  misc_feature: SFT6, sulfotransferase
MSYPNNLSVSPCNTSALLDPKYKAARLIYQQHHPKRRITLYKVAFYCSFGAIIALILNASQMIKITPHESSIDITLYGTVMRPPFSDGKSTPSHTKKNYLHKSSDVFQSTKHKRENEGESTNPTQSNGHSESHLPDSRLTSESSSTLIEDVEEISDRPTVAPDNMGFQQQRSENKIQTANTKVSHSNSERVEFSISDDDVSKRIATTRQVDRRNVDRTAKQPIKTSFKPALPQKDTSKIPLASVKSPVSPIKTSSLKDSSRPRNSMVMQSLPPVQSATALATSSQIQEQVDAQPIDESLLGIGELFADTDENDSDFVLVLPKEEEREDAYSSGYHRRQEIAFAGDSAKRGPENFSLAVSGGTSLEKKSPKLRGKLAIFLHIHKAGGTTMCVMAHYNNEKSLRKSNCNVNDHVDRRELIQGNITTVDRILEKYRVQENYTFLANEWMLPAEIPARKEHMYLTMLRNPLARMESHFFMAMSQLKQGYKRSISKMQCLFTRGMRGAPIPSILDLQRLETNLKVAASARQYFAMNTPDNWQTRSLCGPACAEVPFGMLTEEHLELAKYRLASHFTAVGILEHFKDSITLFHHILHWRRDVSKLFNSHHGTHHKGLTVLETIEKASMKRPKLLTFAKWFHAVNIYDIQLYNFGRTLFQKQAALAGIEVTLGPNLPLDYIAPNGEITSVKDLTQMLTNSVRFRKCHTQCCRSLCAPIGRFWVSTALKLNLVPPRPLCSMGSGNKK

SEQ ID NO: 23 - protein, 496 aa, Aurantiochytrium sp.
  misc_feature: SFT7, putative sulfotranferase
MPWLIKSLARRLADGEISAQELSTKDREPDAVAAVHHGPDLEAAIELEDDRDLLFIHVPRCGGTSLTKLYKVPYKAAAHRGFYHKFGMKYFFYRYGLLEKANFPFFTIENLLVCVSLGISTALWYSGWIKSISALCVNEPGINGFSCPPSLLTIVMYACACLTFLSSTFLFTAPFSGRTDIVRRAYAIIVGQLLCNLTEAEKWLTGVSRKGYVVHFTLAKCIKYGFVQRNELATLESFSVIRNPYSRMVSIYMYNRFGPLESFEHFVECWFKKWKLYKETGDTEEWNIYCHVLPMFEFTHEKQKQIIGCVVKQESLRDITNGALNECFRNDRISTIPPRVLQALKDMPHSNRRKRSKPWQDYYNRRTVRLVYEMYKEDFRIFGYDKHVPGRADLDEALGKDNFDVNSDFIDDLPPQTEAEMYASGRHIRGPDPVSRSSRSIMPHDNLVHAGMLPASASAVSTSSTSSASTASTEPPARSFGGRRNRRSTGSVTPSL

SEQ ID NO: 24 - protein, 546 aa, Aurantiochytrium sp.
  misc_feature: SFT8, putative sulfotransferase
MSIEILAMETNELRGTDSTPDKDGIRELKKDAEQAIGDAEEGLGRHAKKLQGRPQQWRMEAFSKVAGGVRVAARDEACLCGMESPPRSGSSSYEAWTDEDDKEKKPRQNLLKSRETWFLAALLVVATVIIVWLMGPFTSMDSITPSTRGYLSSVKIVTESKENTGYTEDTEEAGTATNIKSDSLTLQKLSSVRDGIKNRCPKGSIIHFLFPHVNKAGGRSMEATFGSDARSRVFPRERYLYHGPRSRTHKFRLTDSHRSYTQLAFNFGWNDSVKELDANSLEFTSHKNCLRWMFGMRDPVSRMVSAFYAITGRTISSPKYGKKPGPHGARHFEHFSCYDKSVGSKRLLDPDFTIEEWARLPLEERERCSPSFNLHVRYLAPEYPDDTPEQLEVAKARLASISWFYIIERMQESWQLFSYVYGTDFVTYTPTFNLNEYSKELSDTARRALEEHNKYDIELYQYAVQLFEERIQIMNRDPTDPFFKPYSFECDTEQICWSKNDARTFWPVSEASWEKHYATPAAAKFLQLCTATRGCWRNDAKDKPFP

SEQ ID NO: 25 - protein, 611 aa, Aurantiochytrium sp.
  misc_feature: SFT9, putative sulfotransferase
MFRNQSDTSRSMSQADGDSQEDVRLVPNGEEKPRTPLGSVGISSRSSSPRALTPSGGSTLASQSNHVDRSPRRSQLIDDGHGSPHAMSQQRAQQQLNGSAGLWSFRVFAIGLLVVSGIALFGMSSSMSMDKTVDENEVDAIRGIPSSSWTALGGSLSAHSKNEVPTLHDSVKKDEFDNTNIKKADNEVDEKESSNDGNGDRGGHSGPIGNSELHHDEKKNNDNYYSSDENTRARKSGKEHHVDHDKSNSEIGDLTEHGSAQIDAKDAPGVSRIRYGPKTIAKCKAGLRHGFSVREDPCLKTGFSKKCGCGSKFTYQIPEAPDSSKALTAKDFRENVVYSYVHVNKAGGQTMKSILMDAIEIGEWSAAGIGTFPGWQAVFKPDDEDRKSAPVYVCGGLHSREVLPDPTQIGECPTRAVWGSLSMGLCELFPDRPCIYFTTVRDPIKRAISEYNYFCVLGEEGRKKWLPEWKRDGECPVNIYEYFMMGRTPANFLKLRLTRGCDEKCGIDVAKANLAHPCLRYIVLEDFKNDIKRFADQVPGALSKALQKAYDEETHMNKSELHPRVEEQIQDKELMAKLASLFEDDIVIYNYALSIRNKKWSTPIQACPAPP

SEQ ID NO: 26 - protein, 554 aa, Aurantiochytrium sp.
  misc_feature: SFT10, putative sulfotransferase
MPWLVHDEEKPEDDLLFVHVPRCGGTSLTKHFEVAKKSRKGLGPWRKFGMLYWWYRYRLLENANFPLVTIENGIFILQYVIAIILFTTIPAYHNDEDCDPSTENCGNGIAAYTLFSSGAMMFLTSTFLATAPIIGRQTFWRRAYALFITYVLCNWTGCEKWLVGCNIKGWFVHFTAAKMLKHGSIKESDLENSFAIVRNPYSRMVSVYMYNRFGPLESFKSFTKRWCKHKLKKYHETGSTSEWDVYCHALPMFEFTHLDGEQIVKCIIKQEELRTLWYPGLTIRSRRHQKRLAEIPPKVAEALQGMPHANSRKRSKPWHDYYDQELVDLVATTYALDFFYFGYDINVPNRPDLKPPPIEPPHDVHPFSSNLDFSIYKDQDLTALARRPSSQFSVSSSPYDSRRDSVSSDFSTKKSPHGPSTATRNRQGSSFSRDHDISEDHVCVEVPQQQLQQHTSSKVLMEEEEGVSREPSLSSKSTSAELTEHVVPVPENLLCGSDESPSSGSSGQPGQHISPIAQQEKSPPVQTSAAARLENDAEGKETEQNGTGASLTET

SEQ ID NO: 27 - protein, 806 aa, Aurantiochytrium sp.
  misc_feature: SFT11, putative sulfotransferase
MMIVSSTGRALLALALLCVLVVVLNLSMTVMEPKLDEEVLKVNEAAWLNRFQLTVPEPSVKPRETGSGTNVKAPIVLVLGIGHQLCEAVAGSFTANRENLRQVIREDEISASKYTLEAVHTIAKYLGSQAQTILPRPADLPQEVAESATVAITRLLDELASSQMTVVAVIPHLVFLWPIVEQQLRLRKLEHVHPIIVVEDGLASSLRDNSHFIDAVGHAMVNERILDTLCLMRVRPFDIEGGTASNLRSAVMQDRIRLAPTVNHNNVEKSPCQVTELASMRNAIIWSKLMRSLALLSLDSMNQIPLLRIPTENVCGFLKENIVFVSSGTSSPLESPSSTINEDQRKKNDRKRRKRADRSPEEGEAKSACEFLGQDVDESKTLSKAFLQDLDLLLDPEVMSSFKFETPVSSRRVLPNPVLESLSNDRIDAGYCVEQANLKKLSMEKRTSLVFPVAHCLPSFLLIGAQKAGTDELAVWLNQLFYARRLDGGVEIHFFDCLGRGKGWNRSPCSRARRPKMLYDSHAGMENLTIARENGQKLAFAWNDLRKESNYVSSWWEKYLELGNMFYTGYKRHAMTFEKTPAYMDLGHPMDMMRMLPSAKLIFMLRDPVQRFISSYFQMCSGMYQTVHNCTYPDMQQRFETLSADFDPTATDRFDQNSVDFFVHRGIAHSLYSYWLMKWRAAFPDQQIMIVFSEHFRVFPQGVIFAIESFLGLTESRQHHQFHPIKTNKGYYVLGGYSKANNPSHKEAPPADLLQQLQAFFNPYSVQLRNLIENSDIVYKPQQQVPGKYNSDFSFPEWLVPASSTL

SEQ ID NO: 28 - protein, 415 aa, Aurantiochytrium sp.
  misc_feature: SFT12, putative sulfotransferase
MSFVEHNSPVEPFTNDREQLLSDKTISVLVDTNSSATRDIPQNLTRRLGYFLHIHKGGGTSVCKSMKAYGENTYKGNCNMGEPHSRANLASGSLATQKKLFDRMLGIGRTFVANEWMLPSQILKQPGLVYITLLRNPLARTESHYAMAMKQAISKIESDSLYASCLWGPQLPDYKTISSKRPRESSRNRIYFARNSPDNWQTRALCGTPCAAVPFGALRKKHLTIAKKNLVRVFDAVGILEQYSESMNLFRNVLQIEVNNTEDLHLGTHHSGESFVDAAIGKAVNSTSVVNFLQWFYAVNLLDIQLYNFGRILFKQQMMYYLNTDVDTGPSLSSNLTLPNGVTYSNQFLSRLLQNYVKKSKCTTTCCGLCAPIGHFWRGVAYRIDMIPTPPECYPTTEPPVMDPEDPEPHVSSFV

SEQ ID NO: 29 - protein, 386 aa, Aurantiochytrium sp.
  misc_feature: SFT13, putative sulfotransferase
MERYSLASLVGALHSPTDETIRPLDENREKIFFVRIQKTGSKSLEYSLRRNVQFLSGVCGSKFNNKHKWLGANPDTCVERGLEVVRSDVRCLTAHHCDFVSAKTTSMESGTTLRYVSLIRHPVVRTLSEARVGCASVYHKGARKRKGPTSGTNNSPRAWDYAYDRLSVVQPVFDCTDDSFMVHFITRPEHSKGMVMRQTKMLTGWNLPGNMEQSIDNYTFHAMKSLDSGERNNAANIAIGVIDAELDIVMVTELYQLSIAVLHRRLGYDIPSYYEEVRERNEEGSIRFGKESKSWKVAEYMHREELLVYKAAIIRLISDAIQLKLTPSPDEVKYILSQVPSRLVPDEWTGRRLPSYRYMCRIHTKRPYNRLGYYVDRICTLHTPVV

SEQ ID NO: 30 - protein, 472 aa, Aurantiochytrium sp.
  misc_feature: SFT14, putative sulfotransferase
MSEYNDARNAHRPPWRSPARTPLLVLSLLCILAIVATQTLTMDASALATLGEPLQSESAIHLNVQKIINTLDTIDATCKETSKISEVDQDPCYSPALRTEIRRCNCRSSMLEANSVTYMIDVKTPQFSSVERVSLDEETIANRLMISFIHINKAGGSTIKKDVIFPSLAEHKWDGAGLGTFRGWQSLGNPWNSRRTLSYMNSIIATAGNEKGMTIEEQGLGGRRSLVSGHDTSGFGGNEFFGDGGSEPLYIRCGKLQPNPKNSFEAENRESGCPLRAIWGSMTMGLCDHFPGRPCSYLVILRDPLERAISNYNYVCIQGAEGRKKWRPDWRKQGFCPLDIREFFEVGLGEPNFLLWRLTRGCDTGCGAQAAIKNLAHPCTRFLLLEELQDGLLHLEQELGSAYGTALRTVRETHATANAAKYGARIETQLANETLMEYLRERLKDDIIIYEAAKKLYKEQWNQPLQSCNSFM

SEQ ID NO: 31 - protein, 870 aa, Aurantiochytrium sp.
  misc_feature: SFT15, putative sulfotransferase
MQRRRHGDLDTNNGDDAGDVVVMDSISASTTPQEPNESAPLMNASSLMSGVFSHNRRMSGRNHPGSGVAKRKIFFWAAMVIAGVILMLIVNHGVQREAPIMSILDSMESVPNGGLGRPGSDTENTLKSIEDKEETNAVNSFEFQNENGNAAKTFEDVESNDASEDLEITQDPEDIRRRCPAGSKIELYYPHLNKGGGRTVDATFFDLDDIGKDPRYRHVSAEPRSKTHKFSMYSYHRTYPALARAKHCLPVPQGLASSCEKTTPELCVRWIYTLREPISRVLSAFYTMTGRKGNTVSGRRPVQKRSAILGGTGAQGDSHFFCRPGSAASQAMQQVDFTIEDWARLPDDERQNCDLAFNIMTKYLAPTTKNNSREQLALAKKRLEDMAWFGVLEQWTESLQLFSYVLGTDLVHYVPTFNLNEYDKNLSPYAKAVLEKHNNLDIELYKYAEELFRSRVAKMRLNQRDPFYKPFSFVCDEEKICWNKNSTEKAWPLTNDPLAHFKSEGEAKEMQLCSPKQGCWRTDVRAPFQKNETEVETKAQKNIKGASLATKLKLPFKARTALQSLETCLPSVFILGARKGGTTSLYEYISAHPRFYGVLLDKKSQSGELLYAHLLDLKRTMSSKTFRKKYNHRFAEELQVSFGSQEGDDSNWMDDIIRGAARTGESTVAYGPACQLPAQIAAACGVNPRIKFIYLVRNPIERIISNYKMRDRLQKAVKGFTIQESIRHDLTSIREAIPTDPQWWTKEDSEGNIPCLYEQDYFNGVWSGMYIVHLTRWMKHYPASQILVLKSEDFLADPAATLRKSLIFIGLDPSVMDVESTVARNYNAAPESAASRSPIPDELRADLQSFYIPYNEALQTTFGIDVSNWN

SEQ ID NO: 32 - protein, 677 aa, Aurantiochytrium sp.
  misc_feature: SFT16, putative sulfotransferase
NSATIGLAKFEAPVMGKAFDRGPMPRHTGRCRQVMVVLLVSLLSILVTRSIEPYLRGGLTEWFEVESITGDLGPDAKPCRESWACGRGESTSSETEPLHKVDVEVREVDPSNTSMEAWKPDRNYGETATKKLNDGGDSEHQYGQIQERAKESVFVKTTNVSSFVSKNASKKISGTEDSEVDNVLTLALASDDLESDIGESHLSNDTDYPSISANSTTENIGKESTEFVLREAVGSVATAEMGDENNYSSEGSENKTSSIASRRDESAKLASPLLSNSQGPSNKSLIDSHGVSIAEDDNATVSLSTEIKNSANTTHEVEGLEGTKEPTGVRFELNTGESKLTTNKVLSPLHVVTELLEKKSTYGAEKALQAPVLVEQIAPLKLYNVPKRNYSFIPESVKESMKKFESPACRAIRYGPSGSALEGTVGISSRAKCSSSMEGTEEALVERGSQILISEAYKLIYVSNMKAASQTLSIVMRERLKAITIGSHKINTYIEQRRAVEPFVKLEDYFVFTFVRDPLSMFYSAYAEIDRRMDRFLKKRTSFQLINRTMKNEPNRILNCLDMVRTGSHLRSDLTPAHMYSQVWKTQRCPGGVGSFLEFDFIGKLENIREDYYALESIIGAKHRPLRIFNGNKQSIYKKHLYNLRTAAFENLEKKACAYSEADYTCFTYQKPTTCQA

SEQ ID NO: 33 - DNA, 20 bp, Artificial Sequence
  Synthetic
  misc_feature: primer oJU-0017
cacgacgttgtaaaacgacg

SEQ ID NO: 34 - DNA, 20 bp, Artificial Sequence
  Synthetic
  misc_feature: primer oJU-0001
gttgtgtggaattgtgagcg

SEQ ID NO: 35 - DNA, 30 bp, Artificial Sequence
  Synthetic
  misc_feature: primer oCAB-0294
tactgctctaggattatttattactaggtc

SEQ ID NO: 36 - DNA, 40 bp, Artificial Sequence
  Synthetic
  misc_feature: primer oCAB-0295
cgtcgttttacaacgtcgtgattgcagaattgacgacgtg

SEQ ID NO: 37 - DNA, 40 bp, Artificial Sequence
  Synthetic
  misc_feature: primer oCAB-0296
cgctcacaattccacacaactttatatgggcatccgctgg

SEQ ID NO: 38 - DNA, 27 bp, Artificial Sequence
  Synthetic
  misc_feature: primer oCAB-0297
tacacgttaaagcatacttctagagaa

SEQ ID NO: 39 - DNA, 21 bp, Artificial Sequence
  Synthetic
  misc_feature: primer oCAB-0298
gcttattgtgaaactcgtgcc

SEQ ID NO: 40 - DNA, 21 bp, Artificial Sequence
  Synthetic
  misc_feature: primer oCAB-0299
gcccctgagctttgtatagtc

SEQ ID NO: 41 - DNA, 25 bp, Artificial Sequence
  Synthetic
  misc_feature: primer PF640
tgtggtatggctgattatgatctag

(19) United States
(12) Patent Application Publication
(10) Pub. No.: US 2020/0399649 A1
(43) Pub. Date: Dec. 24, 2020

(54) ORGANISMS AND METHODS FOR PRODUCING GLYCOMOLECULES WITH LOW SULFATION

(71) Applicant: Conagen Inc., Bedford, MA (US)

(72) Inventors: Nicky C. Caiazza, La Jolla, CA (US); Jun Urano, La Jolla, CA (US)

(73) Assignee: Conagen Inc., Bedford, MA (US)

(21) Appl. No.: 17/012,359

(22) Filed: Sep. 4, 2020

Related U.S. Application Data
(63) Continuation of application No. PCT/US2019/020566, filed on Mar. 4, 2019.
(60) Provisional application No. 62/638,796, filed on Mar. 5, 2018.

(51) Int. Cl. C12N 15/82; C12P 21/00; C07K 16/00; C12N 9/10

(52) U.S. Cl. CPC C12N 15/8258; C12P 21/005; C07K 2317/13; C12N 9/1051; C07K 16/00

(57) ABSTRACT

The invention provides recombinant organisms containing a nucleic acid encoding a heterologous glycomolecule that has a low sulfation profile or that is unsulfated. In one embodiment the heterologous glycomolecule is an immunoglobulin molecule. The recombinant organisms have a genetic modification to at least one sulfotransferase gene, such as a deletion, disruption, or other genetic modification. The cells advantageously produce and, optionally secrete, the heterologous glycomolecule. Thus, the invention provides recombinant organisms that provide glycomolecules having a glycosylation profile that is more similar to the glycosylation profile produced in a mammalian cell, and therefore may be safer and more effective for use as a therapeutic in humans or animals. The glycomolecules can be a glycoprotein, glycopeptide, or a glycolipid.

### CROSS-REFERENCE TO RELATED APPLICATIONS

This application claims benefit of priority under 35 U.S.C. § 119(e) of U.S. Ser. No. 62/638,796, filed Mar. 5, 2018, the entire contents of which is incorporated herein by reference in its entirety.

### INCORPORATION OF SEQUENCE LISTING

The material in the accompanying sequence listing is hereby incorporated by reference into this application. The accompanying sequence listing text file, name SGI2150_1WO_Sequence_Listing.txt, was created on Mar. 1, 2019, and is 111 kb. The file can be accessed using Microsoft Word on a computer that uses Windows OS.

### FIELD OF THE INVENTION

The invention relates to organisms and methods of producing recombinant glycomolecules having no sulfation or reduced sulfation profiles.

### INCORPORATION OF SEQUENCE LISTING

The material in the accompanying sequence listing is hereby incorporated by reference into this application. The accompanying sequence listing text file, name SGI2150_Sequence_Listing.txt, was created on Mar. 5, 2018, and is 111 kb. The file can be assessed using Microsoft Word on a computer that uses Windows OS.

### BACKGROUND OF THE INVENTION

Glycosylated protein and peptide drugs are an important therapeutic resource for the treatment of a variety of diseases and disorders. This class of drugs also includes monoclonal antibodies, which are very useful in many applications. Many glycoprotein or glycopeptide drugs require glycosylation for optimal efficacy in humans and animals. However, different host cells (e.g. mammals, plants, insects, fungi, etc.) produce different glycosylation profiles. This therefore presents safety concerns as the glycosylation profile produced on a glycoprotein or glycopeptide therapeutic produced in non-mammalian host cells could elicit an immunogenic response in a human or animal patient treated with the therapeutic. Therefore, it is advantageous if the glycosylation profiles produced by a non-mammalian host cell on a therapeutic molecule match those produced by mammalian cells. Furthermore, many host cell systems produce polypeptides having sulfated glycan moieties, which is not desirable for some glycoprotein or glycopeptide therapeutics to be used in humans or animals.

Therapeutic glycomolecules are produced in yeasts and fungi. While some engineering in these cell types has been performed to cause these organisms to produce more mammalian-like glycosylation profiles, these organisms are slow growing. While host cell systems that are faster growing are available these produce sulfated glycans, which are not always desirable as some glycoprotein or glycopeptides are safest or most effective in an unsulfated or low sulfation form. It would therefore be of great advantage to have host cell systems that grow quickly and are able to produce therapeutic glycomolecules having N-linked glycosylation profiles similar to what is produced by mammalian cells, and to produce them with fewer or no sulfated glycans.

### SUMMARY OF THE INVENTION

The invention provides recombinant host cells or organisms containing a nucleic acid encoding a heterologous glycomolecule, which is produced by the cell or organism. The glycomolecule can have glycans with a low sulfation profile, or that are unsulfated. In one embodiment the heterologous glycomolecule is an immunoglobulin molecule. The recombinant host cells have a genetic modification in one or more sulfotransferase gene(s). The genetic modification can be a deletion, or another genetic modification that reduces or eliminates expression or activity of the one or more sulfotransferase gene(s). The cells can advantageously produce and, optionally, secrete the heterologous glycomolecule, which can have a glycosylation profile having no sulfated glycans or having fewer sulfated glycans than the same heterologous glycomolecule produced by a corresponding cell that does not comprise the genetic modification. The glycomolecule produced can therefore have a glycosylation profile that is more similar to the glycosylation profile produced in a mammalian cell, and therefore be safer for use as a therapeutic in humans or animals. In various embodiments the glycomolecule can be a glycoprotein, glycopeptide, or glycolipid.

In a first aspect the invention provides a recombinant cell or organism of the Family Thraustochytriaceae that has a nucleic acid encoding a heterologous glycomolecule molecule, and a genetic modification in one or more sulfotransferase gene(s). In various embodiments the genetic modification can be a deletion, a mutation, a disruption, an insertion, an inactivation, an attenuation, or an inversion. In one embodiment the sulfotransferase is a carbohydrate sulfotransferase, for example a member of the Sulfotransferase_2 family (PF03567), or SFT-12, SFT-15, or SFT-16. In one embodiment the cell has the genetic modification in a single gene that encodes a carbohydrate sulfotransferase, which can be any of the sulfotransferases described herein. In some embodiments the cell produces and secretes the heterologous polypeptide molecule or functional portion thereof.

In various embodiments of the invention the heterologous glycoprotein is an immunoglobulin. The heterologous glycomolecule molecule can have fewer sulfated N-glycans relative to a corresponding cell that does not comprise the genetic modification. In various embodiments the heterologous glycomolecule comprises at least 30% or at least 35% or at least 40% unsulfated N-glycans. In one embodiment the ratio of unsulfated to sulfated N-glycans in the heterologous glycomolecule is at least 1:2. The N-glycans can contain Man3-5GlcNAc2. In some embodiments the heterologous glycomolecule molecule is an antibody molecule, or portion thereof. The recombinant cell can be from the taxonomic family Thraustochytriaceae, and in some embodiments is from any of the genera Japanochytrium, Oblongichytrium, Thraustochytrium, Aurantiochytrium, or Schizochytrium.

In various embodiments the recombinant cell or organism can also have a genetic modification to a gene encoding a mannosyl transferase, which in one embodiment is in an alg3 gene.

In various embodiments the heterologous glycoprotein can be any of trastuzumab, eculizurnab, natalizurnab, cetuximab, omalizumab, usteinumab, paniturnumab, and adalimurnab, or a functional fragment of any of them. In various embodiments the immunoglobulin has a low sulfation profile having less than 25% sulfated N-glycans versus the same immunoglobulin produced in a corresponding cell that does not comprise the genetic modification to the one or more sulfotransferase gene(s).

In another aspect the invention provides methods of producing a glycomolecule having a glycosylation profile with low sulfation. The methods involve performing a genetic modification to one or more sulfotransferase genes in a cell or organism of the Family Thraustochytriales, wherein the organism comprises a recombinant nucleic acid encoding a heterologous glycomolecule molecule, or a functional portion thereof, cultivating the cell or organism, and thereby producing the glycomolecule having a glycosylation profile with low sulfation. The methods can also involve a step of providing any recombinant cell or organism described herein. The glycomolecule can be heterologous to the cell or organism, and can be any described herein.

In another aspect the invention provides a therapeutic protein comprising a ratio of S-Man(3-5)/(S-Man(3-5)+Man(3-5) of 0.60 or less. In various embodiments the therapeutic protein can be any immunoglobulin or antibody described herein. The therapeutic protein can be produced by a cell or organism described herein.

In another aspect the invention provides a therapeutic protein or peptide produced by any of the recombinant cells or organisms described herein. In various embodiments the therapeutic protein or peptide can be a glycoprotein or peptide described herein. In some embodiments the therapeutic protein or peptide is an immunoglobulin or antibody described herein.

The summary of the invention described above is not limiting and other futures and advantages of the invention will be apparent from the following detailed description of the invention, and from the claims.

### DESCRIPTION OF THE DRAWINGS

FIG. 1 provides a graphical illustration of amounts of sulfated vs non-sulfated glycans on natalizumab antibody recovered from organisms having the indicated sulfotransferase (SFT) deleted. The total amount of sulfated Man(3-5)GlcNAc2(ManS) and the total amount of non-sulfated Man(3-5)GlcNAc2(Man) as a percentage of all glycans observed are shown in bar graph format.

FIG. 2 provides a graphical illustration of the ratio of S-Man(3-5)/(S-Man(3 5)+Man(3-5)), or sulfated glycans as a percentage of total glycans. Deletion of sulfotransferases resulted in a lower ratio, i.e. less sulfation of glycans.

FIGS. 3A-3B. FIG. 3A provides a plasmid map for pCAB-097. FIG. 3B provides a plasmid map for pCAB-098.

FIGS. 4A-4B, FIG. 4A provides a diagram illustrating an example of a mannose 3 structure. FIG. 4B provides a diagram illustrating an example of a mannose 5 structure.

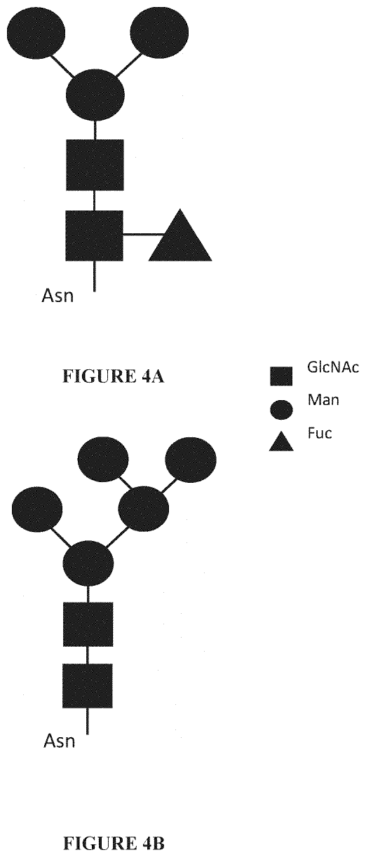

### DESCRIPTION OF THE INVENTION

The invention provides recombinant cells or organisms that contain a nucleic acid molecule encoding a heterologous glycomolecule. The organisms can also have a deletion or other genetic modification in at least one gene encoding a sulfotransferase. The organisms produce the heterologous glycomolecule with a glycosylation profile that contains fewer sulfated glycans versus the same glycomolecule produced by a corresponding organism or host cell that does not contain the deletion or other genetic modification in the at least one gene encoding a sulfotransferase and cultivated under the same conditions. The present inventors discovered unexpectedly that a deletion or other genetic modification of one or more sulfotransferase genes results in a recombinant host cell that produces a heterologous glycomolecule having significantly fewer sulfated glycan moieties. The discovery therefore allows for the production of glycomolecules having a glycan (or glycosylation) profile with low sulfation or no sulfation of glycans. Therefore, the glycomolecule may be safer for use as a therapeutic molecule, and/or less likely to provoke an immune response in a human or other mammal. The glycomolecule may also have higher efficacy in relevant therapeutic applications. In any of the embodiments disclosed herein the glycomolecule can be a glycoprotein, glycopeptide, or glycolipid.

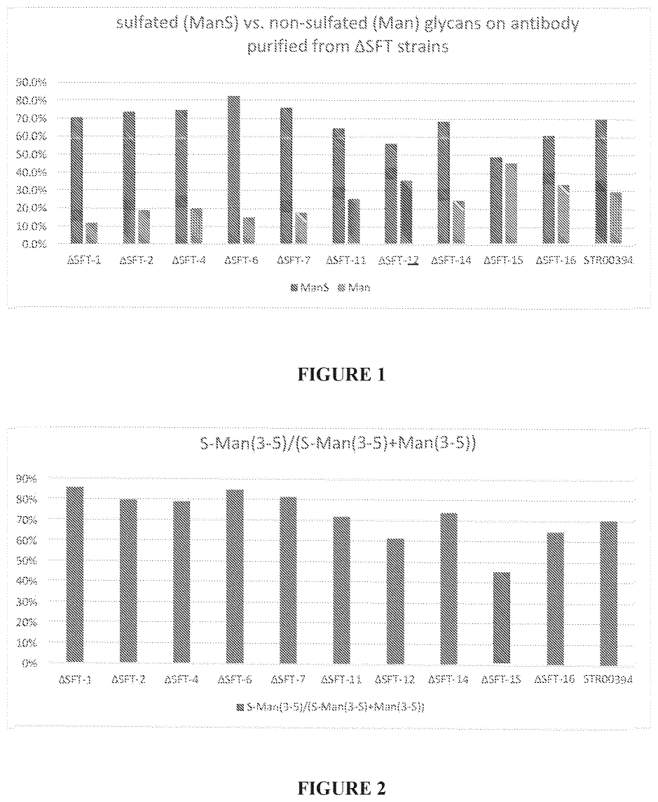

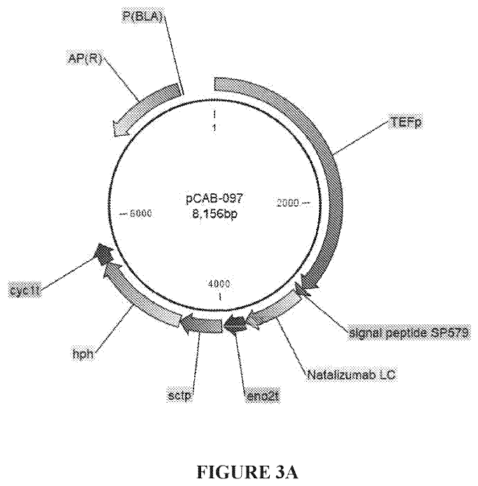

In various embodiments a low sulfation profile of a heterologous glycomolecule produced by a recombinant organism or host cell of the invention is one having 65% or less or 60% or less or 55% or less or 50% or less or 40% or less or 30% or less or 25% or less or 15% or less or 10% or less sulfated glycans (vs. non-sulfated glycans) (FIG. 1). In another embodiment a low sulfation profile for a heterologous glycomolecule can be expressed as having a ratio of sulfated Man(3-5) vs. total sulfated and unsulfated Man(3-5) (which can be expressed as S-Man(3-5)/S-Man(3-5)+Man(3-5)), of 0.65 or less or 0.50 or less or 0.40 or less or 0.30 or less or 0.25 or less or 0.15 or less or 0.10 or less (FIG. 2). In another embodiment a low sulfation profile for a heterologous glycomolecule be one having 30% or more, or 32% or more, or 35% or more, or 40% or more, of unsulfated glycans (vs. sulfated glycans). In any of the N-glycan sulfation profiles the glycan can be mannose(3-5). The sulfation profile can describe sulfation related to the N-glycan profile, the O-glycan profile, the C-linked glycan profile, the phosphoglycosylation profile of a glycomolecule, or any combination or sub-combination of them.

Many proteins or peptides produced by living organisms are modified by glycosylation. Glycoproteins and glycopeptides are proteins or peptides that have carbohydrate groups covalently attached to their polypeptide chain; glycolipids are lipid molecules with a carbohydrate attached by a glycosidic bond. In various embodiments the glycoproteins or glycopeptides can have at least one carbohydrate moiety attached to the polypeptide chain or at least two or at least three or at least four or at least five or at least six or at least seven or at least eight or at least ten carbohydrate moieties attached to at least one polypeptide chain of the glycoprotein, glycopeptide, or glycolipid. The glycan profile can indicate the types of glycans present, their composition and structure, and whether they are sulfated or unsulfated. The glycan (or glycosylation) profile of the glycomolecules can be important for various reasons, such as cellular recognition signals, to prevent an immune response against the protein or peptide, for protein folding, and for stability. Glycosylation can occur to produce any one or more of N-linked glycans, O-linked glycans, C-linked glycans, or phosphoglycosylation, or any combination or sub-combination thereof. N-linked glycosylation refers to the attachment of a sugar molecule (or oligosaccharide known as glycan) to a nitrogen atom, for example an amide nitrogen of asparagine, in the sequence of a protein or peptide. An N-linked glycan (or N-glycan) profile refers to the specific glycosylation (mono- or oligosaccharide) patterns present on a particular glycomolecule, or group of glycoproteins or glycopeptides at a nitrogen atom. The N-glycan profile of a glycomolecule can be a description of the number and structure of N-linked mono- or oligosaccharides that are associated with the particular glycomolecule. O-linked glycosylation refers to the attachment of a sugar molecule to an oxygen atom in an amino acid of a protein or peptide (e.g. serine or threonine). C-linked glycosylation can occur when mannose binds to the indole ring of tryptophan. Phosphoglycosylation occurs when a glycan binds to serine via the phosphodiester bond.

N-glycans and/or O-glycans can also be sulfated (or unsulfated), meaning that they comprise a sulfate moiety (e.g. SO₃) and the amount, extent, or location of sulfation can be part of the N-glycan or O-glycan profile. For certain types of molecules in humans and animals the N-glycan profile does not comprise sulfated N-glycans. It can therefore be desirable that certain therapeutic glycoprotein and glycopeptide molecules produced in host cells not contain sulfated glycans or contain fewer of them, or have a low glycan (or N-glycan) profile. Monoclonal antibodies and other immunoglobulins are just two of many categories of glycoproteins that the invention can be applied to. Thus, in some embodiments the consensus peptide sequence Asn-X-Thr/Ser of a glycomolecule is glycosylated (but not sulfated), where X is any amino acid except proline and Thr/Ser is either threonine or serine.

### Host Cells

In some embodiments the recombinant cells or organisms of the invention are from the Class Labyrinthulomycetes. The Labyrinthulomycetes are single-celled marine decomposers that generally consume non-living plant, algal, and animal matter. They are ubiquitous and abundant, particularly on dead vegetation and in salt marshes and mangrove swamps. While the classification of the Thraustochytrids and Labyrinthulids has evolved over the years, for the purposes of the present application, “Labyrinthulomycetes” is a comprehensive term that includes microorganisms of the Orders Thraustochytrid and Labyrinthulid, and includes (without limitation) the genera Althornia, Aplanochytrium, Aurantiochytrium, Botyrochytrium, Corallochytrium, Diplophryids, Diplophrys, Elina, Japonochytrium, Labyrinthula, Labryinthuloides, Oblongichytrium, Pyrrhosorus, Schizochytrium, Thraustochytrium, and Ulkenia. The recombinant host cells of the invention can also be a member of the Order Labyrinthulales.

In some embodiments the host cell or organism of the invention can be an organism of the Class Labyrinthulomycetes and the taxonomic family Thraustochytriaceae, which family includes but is not limited to any one or more of the genera Thraustochytrium, Japonochytrium, Aurantiochytrium, Aplanochytrium, Sycyoidochytrium, Botryochytrium, Parietichytrium, Oblongochytrium, Schizochytrium, Ulkenia, and Elina, or any combination or sub-combination of them, which is disclosed as if set forth fully herein in all possible combinations. Alternatively, a host Labyrinthulomycetes microorganism can be from a genus including, but not limited to, Aurantiochytrium, Oblongichytrium, and Ulkenia. Examples of suitable microbial species of the invention within the genera include, but are not limited to: any Schizochytrium species, including, but not limited to, Schizochytrium aggregatum, Schizochytrium limacinum, Schizochytrium minutum, Schizochytrium mangrovei, Schizochytrium marinum, Schizochytrium octosporum, and any Aurantiochytrium sp., any Thraustochytrium species (including former Ulkenia species such as U. visurgensis, U. amoeboida, U. sarkariana, U. profunda, U. radiata, U. minuta and Ulkenia sp. BP-5601), and including Thraustochytrium striatum, Thraustochytrium aureum, Thraustochytrium roseum; and any Japonochytrium sp. Strains of Thraustochytriales that may be particularly suitable for the presently disclosed invention include, but are not limited to: Schizochytrium sp. (S31) (ATCC 20888); Schizochytrium sp. (S8) (ATCC 20889); Schizochytrium sp. (LC-RM) (ATCC 18915); Schizochytrium sp. (SR21); Schizochytrium aggregatum (ATCC 28209); Schizochytrium limacinum (IFO 32693); Thraustochytrium sp. 23B ATCC 20891; Thraustochytrium striatum ATCC 24473; Thraustochytrium aureum ATCC 34304); Thraustochytrium roseum (ATCC 28210; and Japonochytrium sp. LI ATCC 28207. In some embodiments the recombinant host cell of the invention can be selected from an Aurantiochytrium or a Schizochytrium or a Thraustochytrium, or all of the three groups together or any combination or sub-combination of them. The recombinant host cells of the invention can also be a yeast cell, such as a yeast selected from any one or more of the genera Saccharomyces, Candida, Pichia, Kluyveromyces, Yarrowia or Arxula. The recombinant host cell of the invention can be selected from any combination of the above taxonomic groups, which are hereby disclosed as every possible combination or sub-combination as if set forth fully herein.

The cells or organisms of the invention can be recombinant, which are cells or organisms that contain a recombinant nucleic acid. The recombinant nucleic acid can encode a functional glycomolecule that is expressed in and, optionally, secreted from the recombinant cell. The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As nonlimiting examples, a recombinant nucleic acid molecule can include any of: 1) a nucleic acid molecule that has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) include conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. A recombinant cell contains a recombinant nucleic acid.

As used herein, “exogenous” with respect to a nucleic acid or gene indicates that the nucleic acid or gene has been introduced (“transformed”) into an organism, microorganism, or cell by human intervention. Typically, such an exogenous nucleic acid is introduced into a cell or organism via a recombinant nucleic acid construct. An exogenous nucleic acid can be a sequence from one species introduced into another species, i.e., a heterologous nucleic acid. A heterologous nucleic acid can also be an exogenous synthetic sequence not found in the species into which it is introduced. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. An exogenous nucleic acid that includes a homologous sequence can often be distinguished from the naturally-occurring sequence by the presence of non-natural sequences linked to the exogenous nucleic acid, e.g., non-native regulatory sequences flanking the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

When applied to organisms, the terms “transgenic” “transformed” or “recombinant” or “engineered” or “genetically engineered” refer to organisms that have been manipulated by introduction of an exogenous or recombinant nucleic acid sequence into the organism, or by the manipulation of native sequences, which are therefore then recombinant (e.g. by mutation of sequences, deletions, insertions, replacements, and other manipulations described below). In some embodiments the exogenous or recombinant nucleic acid can express a heterologous protein product. Non-limiting examples of such manipulations include gene knockouts, targeted mutations and gene replacement, gene replacement, promoter replacement, deletions or insertions, disruptions in a gene or regulatory sequence, as well as introduction of transgenes into the organism. For example, a transgenic microorganism can include an introduced exogenous regulatory sequence operably linked to an endogenous gene of the transgenic microorganism. Recombinant or genetically engineered organisms can also be organisms into which constructs for gene “knock down,” deletion, or disruption have been introduced. Such constructs include, but are not limited to, RNAi, microRNA, shRNA, antisense, and ribozyme constructs. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome, or on a vector or other nucleic acid construct. As used herein, “recombinant microorganism” or “recombinant host cell” includes progeny or derivatives of the recombinant microorganisms of the disclosure. Because certain modifications may occur in succeeding generations from either mutation or environmental influences, such progeny or derivatives may not, in fact, be identical to the parent cell, but are still included within the scope of the term as used herein.

### Genetic Modification

In various embodiments the host cells or organisms of the invention comprise a nucleic acid encoding a heterologous glycomolecule, and a genetic modification in one or more sulfotransferase gene(s) and wherein the host cell produces, and optionally secretes, the encoded heterologous glycomolecule having fewer sulfated glycan moieties compared to the same glycomolecule produced by a corresponding host cell or organism not comprising the genetic modification, or otherwise having a low sulfation profile. In various embodiments the genetic modification can be performed on at one or more or two or more or three or more or four or more or five or more or six or more sulfotransferase genes, or on all sulfotransferase genes of the cell or organism. The glycan moieties on the heterologous glycomolecule can be N-glycan moieties or O-glycan moieties, or both. Thus, in some embodiments the genetic modification to the one or more sulfotransferase gene(s) results in a glycomolecule having fewer sulfated N-glycan moieties, or having fewer sulfated O-glycan moieties, or both, or otherwise having a low sulfation profile. In some embodiments the sulfation is eliminated, or reduced to zero sulfated N-glycan or sulfated O-glycan moieties, or both. A genetic modification denotes any one or more of a deletion, mutation, disruption, insertion, inactivation, attenuation, a rearrangement, an inversion, that results in a physical change to the gene or a regulatory sequence, and that reduces or eliminates expression of the one or more sulfotransferase gene(s) products. An unmodified nucleic acid sequence present naturally in the organism denotes a natural or wild type sequence. In various embodiments the genetic modification can be a deletion. As used herein a deletion can mean that at least part of the nucleic acid sequence is lost, but a deletion can also be accomplished by disrupting a gene through, for example, the insertion of another sequence (e.g. a selection marker), or a combination of deletion and insertion, but a deletion can also be performed by other genetic modifications. A deletion can mean that the gene no longer produces its gene product or, in various embodiments, that the gene produces less than 20% or less than 10% or less than 5% or less than 1% of its gene product versus production without the deletion under standard culturing conditions. The terms deletion cassette and disruption cassette are used interchangeably. In some embodiments N-glycans can have reduced sulfation, low sulfation, or no sulfation as a result of the genetic modification, which N-glycans can include, but are not limited to, Man3GlcNAc2, Man4GlcNAc2 and Man5GlcNAc2, or any combination or sub-combination of them, which are disclosed as if set forth fully herein in all possible combinations. These glycans can be present on a glycomolecule as disclosed herein.

Any genetic modification described herein can be a functional genetic modification, meaning that it causes the modified gene or regulatory sequence to decrease production or activity of the gene product (e.g. an enzyme or encoded polypeptide) by at least 15% or by at least 25% or by at least 50%, or by at least 60%, or by at least 70%, or by at least 80%, or by at least 90% or can eliminate production of the gene product (e.g. the encoded protein or polypeptide). Thus, a deletion or disruption or any genetic modification can also be a functional deletion or disruption. In a gene disruption a functional gene is replaced with one having no activity or with a reduced activity so as to result in a functional genetic modification. A functional genetic modification can be achieved by any of the types of genetic modifications described herein. Thus, a mutation, a deletion, an insertion, an attenuation, a disruption, or any of the other types of genetic modifications described herein, can all be a functional genetic modification as used herein.

In other embodiments an organism of host cell of the invention can have a functional reduction in one or more sulfotransferase gene(s), which can be achieved by the downregulation of a gene or other nucleic acid sequence, or by downregulating the activity of the expressed protein or enzyme. In these embodiments a genetic modification may or may not be present, but the functional reduction is achieved by any mechanism of downregulation of a sulfotransferase gene resulting in less transcription of the gene, or by utilizing a molecule that binds to a sulfotransferase enzyme and reduces or eliminates its activity. Thus, in these embodiments a functional reduction in gene or enzyme activity is achieved by a reduction of activity in the gene product.

### Sulfotransferases

Sulfotransferases catalyze the transfer of a sulfonyl group from an activated sulfate donor onto a hydroxyl group (or an amino group) of an acceptor molecule. In some embodiments the sulfotransferase is a carbohydrate sulfotransferase, which transfers a sulfo group (SO₃H) to a carbohydrate group in a glycomolecule to produce a sulfated carbohydrate group. In one embodiment the acceptor group in the carbohydrate is an alcohol (—OH). Examples of carbohydrates that can be sulfated include, but are not limited to, any one or more of mannose, N-acetylglucosamine, sialic acid, galactose, xylose, and fucose, or any combination thereof. The action of the enzyme can generate carbohydrate sulfate esters. In some embodiments carbohydrate sulfotransferases are transmembrane enzymes in the Golgi that transfer sulfate to carbohydrates on glycoproteins or glycopeptides, for example as they move along the secretory pathway. Their structure can comprise a short cytoplasmic N-terminal, one transmembrane domain, and a large C-terminal Golgi luminal domain.

The one or more sulfotransferases can be encoded by the genome of the cell or organism. There are several examples of carbohydrate sulfotransferases, generally denoted as “CHST”. In some embodiments the carbohydrate sulfotransferase can be a galactose-3-O-sulfotransferase, which can play a role in 3′-sulfation of N-acetyllactosamine in both O- and N-glycans. In various embodiments the sulfotransferase can be a member of the Sulfotransferase_2 family (PFAM PF03567) or any combination or sub-combination thereof, or can be carbohydrate sulfotransferase 6 (CHST6), a CHST8, or CHST9, or CHST10, or CHST11, or CHST12, or CHST13, or a D4ST1, which transfers sulfate to position 4 of the GalNAc residue of dermatan sulfate, or any combination or sub-combination of these promoters. The one or more sulfotransferase(s) can be from the Sulfotransferase_2 family, which includes but is not limited to chondroitin 6-sulfotransferase, heparan sulfate 2-O-sulfotransferase (HS2ST), heparan sulfate 6-sulfotransferase (HS6ST). Any combination or sub-combination of the sulfotransferases described herein can contain a genetic modification in a cell or organism of the invention, as described herein. The recombinant cells or organisms of the invention can have the genetic modification in one or two or three or four or five or six or more than six or in all sulfotransferase genes of the cell or organism. The one or more sulfotransferase gene(s) can be present as more than one copy and in one embodiment the cells and methods of invention can involve performing the genetic modification on more than one or on all copies of the gene(s).

Sulfotransferases that can advantageously be deleted, disrupted, or subjected to another genetic modification in the invention include those of the PFAM family PF03567 (Sulfotransferase_2). In some embodiments a sulfotranferase advantageously subjected to genetic modification according to the invention has a homology to a member of the PFAM family PF03567, or to a member of the Sulfotransferase_2 family. The homology can be a sequence identity of at least 20% or at least 30% or at least 40% or at least 50% or at least 60% or at least 70% or at least 80% or at least 90% or at least 95% or at least 96% or at least 97% or at least 98% or at least 99% to said member of said family. In some embodiments a PFAM domain can also be a molecule having an E-value of 1×10⁻⁴ or less. In other embodiments the sulfotransferase subjected to or having the genetic modification is a sulfotransferase of SEQ ID NO: 1-41 or a sulfotransferase having a sequence identity of at least 20% or at least 30% or at least 40% or at least 50% or at least 60% or at least 70% or at least 80% or at least 90% or at least 95% or at least 96% or at least 97% or at least 98% or at least 99% to a sulfotransferase of SEQ ID NO: 1-41 or to any two or more, or three or more, or four or more, or five or more sulfotransferase(s) of SEQ ID NO: 1-41, or to any combination or sub-combination of them, which is hereby disclosed as each possible sub-combination as if set forth fully herein. The sulfotransferase can also be any described herein from any of the host cell organisms or cells described herein. In additional embodiments the sulfotransferase can be any the sulfotransferases CHST6 (EC 2.8.2), a CHST8, or CHST9, or CHST10, or CHST11, or CHST12, or CHST13, or D4ST1, or can have a sequence identity of at least 20% or at least 30% or at least 40% or at least 50% or at least 60% or at least 70% or at least 80% or at least 90% or at least 95% or at least 96% or at least 97% or at least 98% or at least 99% to any of them, or to any combination or sub-combination of them, which is hereby disclosed as each possible sub-combination as if set forth fully herein.

It was discovered that the deletion, disruption, or other genetic modification as described herein of one or more sulfotransferase gene(s) in a Labyrinthulomycete (e.g. an organism of the family Thraustochytriales) resulted in production of a heterologous glycomolecule having a glycan profile (e.g. an N-glycan or O-glycan profile) that contains no sulfated glycans or fewer sulfated glycans compared with the same heterologous glycomolecule produced by a corresponding organism grown under the same conditions that does not have the genetic modification, or that otherwise has a low glycan profile. In various embodiments the genetic modification can be a deletion, knock out, or disruption of the sulfotransferase.

### Heterologous Glycomolecules

In some embodiments the heterologous glycomolecule produced by the cells or organisms of the invention can be a therapeutic molecule, such as a glycoprotein, glycopeptide, or glycolipid, e.g. enzymes, Ig-Fc-Fusion proteins, or an antibody. The antibody can be a functional antibody or a functional fragment of an antibody. In various embodiments the antibody can be alemtuzumab, denosumab, eculizumab, natalizumab, cetuximab, omalizumab, ustekinumab, panitumumab, trastuzumab, belimumab, palivizumab, natalizumab, abciximab, basiliximab, daelizumab, adalimumab (anti-TNF-alpha antibody), tositumomab-I131, muromonab-CD3, canakinumab, infliximab, daclizumab, tocilizumab, thymocyte globulin, anti-thymocyte globulin, or a functional fragment of any of them. The glycoprotein can also be alefacept, rilonacept, etanercept, belatacept, abatacept, follitropin-beta, or a functional fragment of any of them. The antibody can also be any antiTNF-alpha antibody or an anti-HER2 antibody, or a functional fragment of any of them. The glycoprotein can be an enzyme, for example idursulfase, alteplase, laronidase, imiglucerase, agalsidase-beta, hyaluronidase, alglucosidase-alfa, GalNAc 4-sulfatase, pancrelipase, or DNase. Each of these proteins is an antibody and/or a therapeutic protein, and can also be a monoclonal antibody. A functional antibody (or immunoglobulin) or fragment of an antibody binds to a target epitope and thereby produces a response, for example a biological response or action, or the cessation of a response or action. The response can be the same as the response to a natural antibody, but the response can also be to mimic or disrupt the natural biological effects associated with ligand-receptor interactions.

When the protein is a functional fragment of an antibody it can comprise at least a portion of the variable region of the heavy chain, or can comprise the entire antigen recognition unit of an antibody, but nevertheless comprise a sufficient portion of the complete antibody to perform the antigen binding properties that are similar to or the same in nature and affinity to those of the complete antibodies. In various embodiments a functional fragment of a glycoprotein, glycopeptide, glycolipid, antibody, or immunoglobulin can comprise at least 10% or at least 20% or at least 30% or at least 50% or at least 60% or at least 70% or at least 80% or at least 90% or at least 95% of the native sequence, and optionally can also have at least 70% or at least 80% or at least 90% or at least 95% sequence identity to that indicated portion of the native sequence; for example, a functional fragment can comprise at least 85% of the native antibody sequence, and have a sequence identity of at least 90% to that portion of the native antibody sequence. Any of the recombinant cells disclosed herein can comprise a nucleic acid encoding a functional and/or assembled antibody molecule described herein, or a functional fragment thereof.

In various embodiments the glycomolecule can be a hormone, e.g., human growth hormone, leutinizing hormone, thyrotropin-alpha, interferon, darbepoetin, erythropoietin, epoetin-alpha, epoetin-beta, FS factor VIII, Factor VIIa, Factor IX, anithrombin/ATIIcytokines, clotting factors, insulin, erythropoietin (EPO), glucagon, glucose-dependent insulinotropic peptide (GIP), cholecystokinin B, enkephalins, and glucagon-like peptide (GLP-2) PYY, leptin, and antimicrobial peptides. In any of the embodiments the glycomolecule can be encoded on DNA exogenous to the cell, e.g. a plasmid, artificial chromosome, other extranuclear DNA, or another type of vector DNA. It can also be present on an exogenous sequence inserted into the cellular genome.

As used herein, the terms “percent identity” or “homology” with respect to nucleic acid or polypeptide sequences are defined as the percentage of nucleotide or amino acid residues in the candidate sequence that are identical with the known polypeptides, after aligning the sequences for maximum percent identity and introducing gaps, if necessary, to achieve the maximum percent homology. N-terminal or C-terminal insertion or deletions shall not be construed as affecting homology, and internal deletions and/or insertions into the polypeptide sequence of less than about 30, less than about 20, or less than about 10 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q 10 (gap creation penalty); R 10 (gap extension penalty); wink 1 (generates word hits at every winkth position along the query); and gapw 16 (sets the window width within which gapped alignments are generated). The equivalent Blastp parameter settings for comparison of amino acid sequences can be: Q 9; R 2; wink 1; and gapw 32. A BESTFIT® comparison between sequences, available in the GCG package version 10.0, can use DNA parameters GAP 50 (gap creation penalty) and LEN 3 (gap extension penalty), and the equivalent settings in protein comparisons can be GAP 8 and LEN 2.

When referring to the heterologous glycomolecules or nucleic acid sequences of the present disclosure, included in the disclosure are sequences considered to be derived from the original sequence, which have sequence identities of at least 40%, at least 45%, at least 50%, at least 55%, of at least 60, %, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% or 85-99% or 85-95% or 90-99% or 95-99% or 97-99% or 98-99% sequence identity with the full-length polypeptide or nucleic acid sequence. Fragments of sequences can have a consecutive sequence of at least 30, at least 50, at least 75, at least 100, at least 125, 150 or more, or 30-50 or 30-75 or 30-100 amino acid residues of the entire protein, or at least 100 or at least 200 or at least 300 or at least 400 or at least 500 or at least 600 or at least 700 or at least 800 or at least 900 or at least 1000 or 100-200 or 100-500 or 100-1000 or 500-1000 or any of these amounts but less than 500 or less than 1000 or less than 2000 consecutive nucleotides. Also disclosed are variants of such sequences, e.g., wherein at least one amino acid residue has been inserted N- and/or C-terminal to, and/or within, the disclosed sequence(s) which contain(s) the insertion and substitution. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme).

### Promoters and Terminators

The recombinant cell or organism of the invention can be any suitable organism but in some embodiments is a Labyrinthulomycetes cell or a Thraustochytriaceae cell or other cell described herein. Promoters and terminators can be used on expression cassettes or other nucleic acid constructs in the invention, and the promoter (and terminator) can be any suitable promoter and/or terminator. Promoters and/or terminators disclosed herein can be used in any combination or sub-combination. For example, any promoter described herein (or other promoters that may be isolated from or functional in the host cell or organism), or derived from such sequences, can be used in combination with any terminator described herein or other terminators functional in the recombinant cell or organism, or derived from such sequences. For example, terminator sequences may be derived from organisms including, but not limited to, Heterokonts (including Labyrinthulomycetes), fungi, microalgae, algae, and other eukaryotic organisms. In various embodiments the promoter and/or terminator is any one operable in a cell or organism that is a Labyrinthulomycetes cell, including any Family or genus thereof. Any of the constructs can also contain one or more selection markers, as appropriate. A large number of promoters and terminators can be used with the host cells of the invention. Those described herein are examples and the person of ordinary skill with resort to this disclosure will realize or be able to identify other promoters useful in the invention. Examples of promoters that can be utilized in the invention include the alphatubulin promoter, TEF, TEF1, hsp60, hsp60-788 promoter, hsp70, RPL11, Tsp-749 promoter, Tubu738 promoter, Tubu-997 promoter, a promoter from the polyketide synthase system, and a fatty acid desaturase promoter. Examples of useful terminators include pgk1, CYC1, and eno2. Promoters and terminators can be used in any advantageous combination and all possible combinations of these promoters and terminators are disclosed as if set forth fully herein.

In some embodiments the expression cassettes utilized in the invention comprise any one or more of 1) one or more signal sequences; 2) one or more promoters; 3) one or more terminators; and 4) an exogenous sequence encoding one or more proteins, which can be a heterologous protein; 4) optionally, one or more selectable markers for screening on a medium or a series of media. These components of an expression cassette can be present in any combination, and each possible sub-combination is disclosed as if fully set forth herein. In specific embodiments the signal sequences can be any described herein, but can also be other signal sequences. Various signal sequences for a variety of host cells are known in the art, and others can be identified with reference to the present disclosure and which are also functional in the host cells. In exemplary specific embodiments the promoter can be an alpha-tubulin promoter or TEFp, with alpha-tubulin promoter being the weaker of the two. The promoters can be paired with any suitable terminator, but in specific embodiments the tub-alpha-p can be paired with the pgk1. In another embodiment the TEFp promoter can be paired with the eno2 terminator, both terminators being from Saccharomyces cerevisiae and also being functional in Labyrinthulomycetes. The selectable marker can be any suitable selectable marker or markers but in specific embodiments it can be nptII or hph. In one embodiment nptII can be linked to the heavy chain constructs and hph can be linked to the light chain constructs.

The present invention also provides a nucleic acid construct, which is a deletion or disruption cassette for performing a deletion, knock out, disruption, or other genetic modification in a gene that encodes a sulfotransferase. The nucleic acid construct can be regulated by a promoter sequence and, optionally, a terminal sequence functional in a host cell. The host cell can comprise an expression cassette and also a deletion, knock out, or disruption cassette as disclosed herein, which can also be a CRISPR/Cas 9 cassette that can delete any one or more of the target genes as disclosed herein. In any of the embodiments the host cell can be a Labyrinthulomycetes cell or organism, for example a cell or organism of the family Thraustochytriceae, such as any of the genera Aurantiochytrium, a Schizochytrium, or a Thraustochytrium. The construct or cassette can also have a sequence encoding 5′ and 3′ homology arms to the gene encoding a sulfo transferase, which in some embodiments can be a 1,3-sulfo transferase. The construct can also have a selection marker, which in one embodiment can be nat, but any appropriate selection marker can be used.

### Additional Modifications

In some embodiments the recombinant cells or organisms of the invention contain a genetic modification in addition to the genetic modification to one or more gene(s) encoding a sulfotransferase, as described herein. In one embodiment the additional genetic modification can be a deletion, disruption, or other genetic modification described herein in one or more gene(s) that encode a mannosyl transferase enzyme. As a result of the additional modification the cells produce a glycomolecule that has an N-linked glycan profile that is more humanized or human-like, or is simplified. In some embodiments the glycomolecule has at least 25% or at least 35% or at least 45% or at least 55% or at least 70% or at least 80% fewer high mannose N-glycan structures than the same molecule produced by a corresponding cell that does not have the modification to the one or more mannosyl transferase gene(s). The glycomolecule can also have a low sulfation profile, as described herein. The genetic modification can be in any one or more of the alg3 gene(s) or in any one or more gene(s) in the mannosyl transferase gene family, or in a regulatory sequence affecting expression of the gene (e.g. in a promoter), but can also be in a non-regulatory sequence. Members of this family include, but are not limited to, alg1, alg2, alg3, alg6, alg8, alg9, alg10, alg11, alg13, and alg14. The deletion or knockout or other genetic modification can be present in any one or more genes of the mannosyl transferase gene family, or in any combination or sub-combination of them. The host cell can be a cell of the invention described herein. Therefore, the proteins produced avoid many of the problems associated with the use of glycoproteins, glycopeptides, or glycolipids having patterns of glycosylation of non-human species. When combined with the modification to the one or more genes encoding a sulfotransferase as described herein, further benefit is realized by further humanizing the glycomolecule by reducing or removing sulfate moieties on the N-glycan structures.

### Methods

The invention also provides methods of producing glycomolecules in host cells described herein that have a glycan profile having low sulfation of N-glycans or O-glycans, or both as described herein. The methods can involve any one or more steps of: transforming or obtaining a host cell with an expression vector or other exogenous nucleic acid encoding a heterologous glycomolecule for expression from the vector or from integration into the chromosome of the cell, a step of performing a genetic modification to one or more sulfotransferase gene(s) as described herein (e.g. by transforming with a deletion or disruption construct), cultivating the cell, and harvesting the heterologous glycomolecule that has a glycan profile with low sulfation as described herein. Optionally the method can also have a step of performing a deletion or other genetic modification to one or more mannosyl transferase genes as described herein.

In one embodiment the method involves transforming the host cell with a deletion cassette, knock out cassette, or disruption or other cassette to thereby perform the genetic modification on one or more gene(s) that encodes a sulfotransferase as disclosed herein, cultivating the cell, and harvesting a glycomolecule that has a low sulfation glycan profile as described herein.

The invention also provides methods of producing a glycomolecule described herein. The methods involve providing a recombinant Labyrinthulomycete cell that produces a heterologous glycomolecule and that produces or has a sulfotransferase enzyme, and contacting the recombinant cell with a molecule that reduces sulfotransferase enzyme activity in the cell to thereby produce the glycomolecule having a low sulfation glycan profile, which can be any as described herein.

The invention also provides a method of producing a glycomolecule having a glycan profile as disclosed herein. The method involves providing a recombinant Labyrinthulomycete cell that produces a heterologous glycomolecule, modifying the Labyrinthulomycete cell to reduce the activity of or inactivate at least one sulfotransferase enzyme of the cell, and producing the glycomolecule. The cell can also have a genetic modification to one or more mannosyl transferase genes as described herein. Modifying the cell can involve disrupting or deleting or otherwise genetically modifying a gene encoding one or more sulfotransferase enzyme(s), as described herein. In various embodiments the cell is modified by inactivating the transcription or translation of a gene encoding one or more sulfotransferase enzyme(s), or by contacting the Labyrinthomycete cell with an inhibitor of sulfotransferase. In another embodiment the sulfotransferase enzyme can be inactivated by contacting the cell with antisense RNA, RNAi, or a ribozyme. The one or more sulfotransferase enzyme(s) can also be inactivated by a transcriptional regulator. The inhibitor can be produced by one or more nucleic acid molecules comprised in the cell or by any method described herein. And the inhibitor can be any described herein.

### Enzyme Inhibition

In some embodiments the activity of the sulfotransferase can be inhibited, reduced, or eliminated through the use of RNA interference (RNAi) to inhibit the expression of one or more genes encoding a sulfotransferase. The sulfotransferase inhibited can be any as described herein. The inhibition can involve mutating the sulfotransferase gene, or can be a separate gene that, when expressed, binds to the enzyme or otherwise causes a reduction in activity of the enzyme. The RNAi suppression of a gene can be accomplished by methods known in the art including, but not limited to, the use of antisense RNA, a ribozyme, small interfering RNA (siRNA) or microRNA (miRNA). The siRNA or miRNA can be transcribed from a nucleic acid inserted into the genome of the cell, or can be transcribed from a plasmid or other vector transformed into the cell, or can be provided in a growth medium in which the cell is comprised.

In other embodiments the activity of the sulfotransferase enzyme can be inhibited by the use of an enzyme inhibitor. The inhibitor can be a sulfation inhibitor, and can be an inhibitor of sulfotransferase. In various embodiments the inhibitor can be chlorate, brefeldin A, or a galactosamine compound, or another sulfotransferase inhibitor. The enzyme inhibitors can be produced by nucleic acids inserted into the genome of the cell, or can be produced from nucleic acids present on a plasmid or other vector transformed into the cell, or can be included in a growth medium in which the cell is grown. The inhibitor can also be an antibody directed to one or more epitopes on the enzyme, or on a substrate for the enzyme.

### Compositions

The present invention also provides compositions having a glycomolecule produced by a recombinant cell or organism described herein, wherein the glycomolecule has a glycan profile with no sulfated glycans or with a low sulfation profile, i.e. the glycomolecule has 65% or less or 50% or less or 40% or less or 30% or less or 25% or less or 15% or less or 10% or less sulfated glycans vs. non-sulfated glycans. In another embodiment the glycomolecule has a glycan profile having a ratio of sulfated Man(3-5) vs. total sulfated and unsulfated Man3-5 (or S-Man(3-5)/S-Man(3-5)+Man(3-5)), of 0.65 or less or 0.50 or less or 0.40 or less or 0.30 or less or 0.25 or less or 0.15 or less or 0.10 or less.

The glycan profile can be an N-glycan profile, an O-glycan profile, or both. The composition can be produced by and derived from a recombinant Labyrinthulomycete cell or organism described herein. Derived from a cell means that the glycomolecule was synthesized by the cell. In some embodiments the entire glycomolecule was synthesized by the cell, including the glycan portion. The cell can comprise a genetic modification in one or more genes that encode a sulfotranferase, and optionally in one or more mannosyl transferase genes, as described herein. The composition can be any of the compositions derived from host cells, as described herein.

The present invention also provides compositions containing a therapeutic glycomolecule produced by the cells or organisms of the invention described herein. The therapeutic glycomolecule can be one useful for therapy in a human or animal patient. The therapeutic glycomolecule contained in the composition can be any described herein, for example an antibody, an immunoglobulin, a single domain antibody, or any therapeutic protein described herein. The therapeutic glycomolecule can be provided in a pharmaceutically acceptable carrier.

### Glycosylation and Analysis

Glycan analysis can be performed to determine the identity, structure, and/or quantity of carbohydrates present on a glycomolecule as well as the site of modification. Glycan analysis permits the determination of and/or relative quantities of glycans present. In various embodiments glycans that may be present (e.g. when the glycoprotein is an antibody), and which may be sulfated or unsulfated according to the invention include but are not limited to Man3GlcNAc2, Man4GlcNAc2 and Man5GlcNAc2.

Persons of ordinary skill understand methods of releasing glycans from a glycomolecule, which can include enzymatic release. One example of enzymatic release includes the use of peptide-N-glucosidase F (PNGaseF) or Endo H, which generally releases most glycans. PNGaseA can be used to release glycans that contain alpha1-3 linked fucose to the reducing terminal GlcNAc. O-glycans can be released using chemical methods (e.g. beta-elimination).

High performance anion exchange chromatography with derivatization-free, pulsed amperometric detection (HPAE-PAD) is a method known by persons of ordinary skill in the art for the separation and analysis of glycans. In this technique glycans are separated based on various criteria (including size and structure) and a glycan profile can be generated. Mass spectrometry and HPLC are other techniques used for the analysis of glycans and the generation of a glycan profile.

The host cells or organisms of the invention produce a glycomolecule having a low sulfation profile as disclosed herein, or that the glycoproteins, glycopeptides, or glycolipids produced have 50% or less or 35% or less or 25% or less or 15% or less sulfated glycan moieties compared to the same product produced by a corresponding organism that does not have the genetic modification and grown under the same conditions. A low sulfation profile can also mean that the glycoproteins or glycopeptides produced in the host cells or organisms of the invention have at least 1% or at least 10% sulfated glycan moieties.

### Organisms

Persons of ordinary skill in the art are able to isolate Labyrinthulomycetes organisms described herein in various coastal marine habitats, such a salt marshes and mangrove swamps (e.g. found in tropical regions). For the present invention cells of the taxonomic family Thraustochytriaceae (Aurantiochytrium sp.) were isolated from a sample obtained from a mangrove lagoon using a plankton tow (10 um). Organisms harvested were cultured on a media containing sea water, glucose, yeast extract and peptone, and standard enrichment steps were carried out on the same media. A single colony isolate was selected that was found to be amenable to producing and secreting proteins and was used as the base strain (designated #6267).

### Example 1

### Synthesis of Constructs for Producing Heterologous Glycomolecule

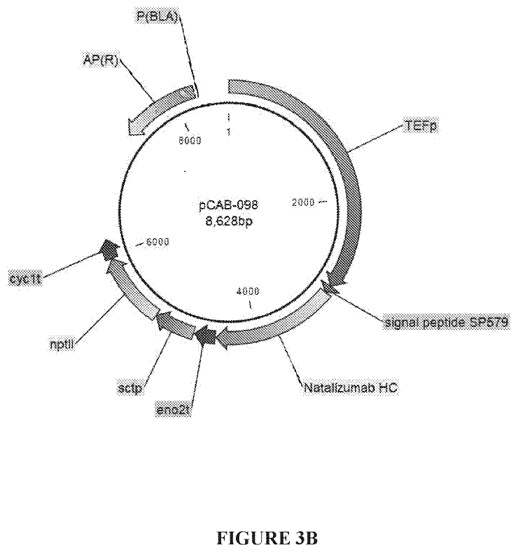

Natalizumab is a humanized IgG4k monoclonal antibody useful in the treatment of multiple sclerosis and Crohn's disease. Natalizumab expression constructs pCAB097 & 098 (FIGS. 3a and 3b) were synthesized as follows. Constructs pCAB097 is an expression cassette with the TEF promoter (SEQ ID NO: 1) driving expression of the natalizumab light chain (SEQ ID NO: 3) where secretion is mediated by signal peptide #579 (SEQ ID NO: 2). This cassette carries the hph marker for selection.

Constructs pCAB098 is an expression cassette with the TEF promoter driving expression of the natalizumab heavy chain (SEQ ID NO: 4) where secretion is mediated by signal peptide #579 (SEQ ID NO: 2). This cassette carries the nptII marker for selection.

TABLE 1
Description of natalizumab expression constructs.

| Construct | Promoter | Signal peptide | Gene | Marker |
|---|---|---|---|---|
| pCAB097 | TEF | SP579 (SEQ ID NO: 2) | natalizumab | hph |
| | | | light chain | |
| pCAB098 | TEF | SP579 (SEQ ID NO: 2) | natalizumab | nptII |
| | | | heavy chain | |

Table of Strains
#6267—base Aurantiochytrium sp. strain
#6602—natalizumab-producing strain
#6920 natalizumab-producing and carrying Cas9
#7087 natalizumab-producing, carrying Cas9, alg3 deletion, nat* marker
#0394 (also called 7534) natalizumab-producing, Cas9, alg3 deletion, bsr marker
#9156 same as #0394 with ku70 deleted
* nourseothricin resistance gene

### Example 2

### Construction of Natalizumab-Producing Strain #6602

Strains expressing natalizumab were produced by co-transforming the Aurantiochytrium sp. base strain #6267 with pCAB097 and 098 described above that had been linearized by BsaI digestion. Five transformants (clones #3, 14, 15, 20 & 31) were resistant to both hygromycin B and paromomycin and were screened by ELSA for production of antibody. Each clone was cultured overnight in 3 mL FM002 (17 g/L Instant Ocean®, 10 g/L yeast extract, 10 g/L peptone, 20 g/L dextrose) in a 24-well plate. They were then diluted 1000× into fresh FM002 (2.5 mL) and incubated for about 24 hours. The cells were pelleted by centrifugation (2000 g×5 min) and the supernatants assayed for the presence of antibody by HC-capture/LC-detect sandwich ELISA. All five clones produced detectable antibody. These clones were again cultured as described and accurate titers were obtained using a commercially available human IgG subclass profile kit. These titers are shown in Table 2. Clone #31 was given the ID #6602 and used as a natalizumab strain for further work.

TABLE 2
Summary of natalizumab titers in clones
transformed with pCAB097 & 098.

| strain | titer (mg/L) |
|---|---|
| natalizumab #03 | 44 |
| natalizumab #14 | 54 |
| natalizumab #15 | 49 |
| natalizumab #20 | 30 |
| natalizumab #31 | 95 |

### HC-Capture/LC-Detect Sandwich ELISA

Monoclonal antibody (mAb) was measured using a sandwich ELISA. mAb was captured using a mouse anti-Human IgG Fc antibody adsorbed onto Nunc™ MaxiSorp™ plates. Plates coated with the capture antibody were washed 5× with wash buffer (1×PBS, 0.05% Tween20). Samples (200 μL) were added and incubated at 37° C. for 1 hour. A dilution series of human IgG1 Kappa was also applied to generate a standard curve. The plates were again washed 5× with wash buffer and detection antibody (goat Anti-Human Kappa-HRP) diluted 20,000× in dilution buffer (1×PBS, 0.1% Tween20, 5% bovine calf serum (BCS)) was added. After incubation at 37° C. for 1 hour, the plate was washed 5× with wash buffer. The HRP was detected using 1-Step™ Ultra TMB-ELISA Substrate solution. The plates were read over time at 650 nm. Alternatively, stop solution (2N H₂SO₄) was applied and the plates read at 450 nm. When used for screening, the measured absorbances were used. When used to assess titers, a standard curve is generated based on the dilution series of the standard human IgG1 Kappa antibody and used to interpolate titers for the unknown samples.

Cas9 Expression Constructs: pAM-001

Constructs pAM001 (SEQ ID NO: 5) is an expression cassette for Cas9. This cassette carries sequences for the constitutive expression of Cas9 from Streptococcus pyogenes under the control of the hsp60 promoter (SEQ ID NO: 6). This construct also carries the TurboGFP reporter and the ble marker.

### Example 3

### Construction of Natalizumab Producing Strain Carrying Cas9, #6920

Cas9 was introduced into the natalizumab base strain by transforming this strain with the cassette pAM-001 linearized by digestion by AhdI. Zeocin™ resistant colonies were examined for presence of the cassette by GFP fluorescence on a Typhoon™ FLA 9000 laser scanner. These natalizumab+Cas9 transformants producing GFP were screened for the presence of the Cas9 expression cassette by amplification of an appropriately sized product by PCR using primers oSGT-JU-1360 and PF640 (SEQ ID Nos: 7 and 41). These primers would amplify the Cas9 expression cassette from the 3′ end of the hsp60 promoter to the 5′ end of the sv40 terminator. Ten positive clones were examined for production of natalizumab by ELISA. Clones were cultured overnight in 3 mL FM002 (17 g/L Instant Ocean®, 10 g/L yeast extract, 10 g/L peptone, 20 g/L dextrose) in a 24-well plate. 10 μL of this culture was used to inoculate fresh FM002 (3 mL) and incubated for about 24 hours. The cells were pelleted by centrifugation (2000 g×5 min) and the supernatants assayed for the presence of antibody by HC-capture/LC-detect sandwich ELISA. The results are shown in Table 3. Clone 3 was chosen to be used for further development as the natalizumab Cas9 clone and given the ID #6920.

TABLE 3
Natalizumab titers produced by clones transformed with Cas9.

| Sample | natalizumab titers (mg/L) |
|---|---|
| #6602 + Cas9 Clone 1 | 0 |
| #6602 + Cas9 Clone 2 | 44.54 |
| natalizumab + Cas9 Clone 3 | 61.05 |
| natalizumab + Cas9 Clone 4 | 47.75 |
| natalizumab + Cas9 Clone 5 | 41.11 |
| natalizumab + Cas9 Clone 6 | 15.80 |
| natalizumab + Cas9 Clone 7 | 26.99 |
| natalizumab + Cas9 Clone 8 | 0 |
| natalizumab + Cas9 Clone 9 | 57.95 |
| natalizumab + Cas9 Clone 10 | 28.71 |
| nat. base strain #6602 | 84.52 |
| nat. base strain #6602 | 84.62 |

### Example 4

### Construction of alg3 Deletion Cassette

The disruption cassette utilized was a linear fragment of DNA having three parts, from 5′ to 3′: 1) a 5′ homology arm, 2) a selection marker and 3) a 3′ homology arm. The 5′ homology arm can be a region of 500 1000 bp found upstream in the genome of the sequence being targeted for deletion. Selection markers generally contain a sequence encoding for expression of a gene (i.e. an antibiotic resistance gene) that allows for selection of successful transformants. The 3′ homology arm can be a region of 500 1000 bp found downstream in the genome of the sequence being targeted for deletion.

This example describes the construction of a disruption cassette of the alg3 gene in Aurantiochytrium sp. Three translation IDs (SG4EUKT579099, SG4EUKT579102, and SG4EUKT561246) (SEQ ID Nos: 11-13, respectively) were found in the genome assembly of the Aurantiochytrium sp. base strain (#6267). All three sequences encode a 434 amino acid protein (mannosyl transferase) (SEQ ID Nos: 8-10). SG4EUKT579099 and SG4EUKT579102 are identical at both the amino acid and nucleotide levels. SG4EUKT561246 shares greater than 99% identity to the other sequences at both the amino acid and nucleotide levels. This high level of identity allowed for the targeting of all three sequences using Cas9 and a single guide RNA (gRNA) sequence (SEQ ID NO: 14) as well as a single disruption cassette (alg3::nat) comprised of a selectable marker (nat) providing resistance to nourseothricin that is flanked by 5′ and 3′ alg3 homology arms (500-about 1000 bp).

The alg3::nat disruption cassette was generated by amplifying the 5′ and 3′ alg3 homology arms from the base strain (#6267) genomic DNA, while the selectable marker (nat) was amplified from nat containing plasmid DNA (pSGI-JU-97). The nat marker was amplified using primers oSGI-JU-0017 (SEQ ID NO: 33) and oSGI-JU-0001 (SEQ ID NO: 34). The 5′ homology arm was amplified using primers oCAB-0294 (SEQ ID NO: 35) and oCAB-0295 (SEQ ID NO: 36), the latter has a 5′ extension that is complementary to oSGI-JU-017. The 3′ homology arm was amplified using primers oCAB-0296 (SEQ ID NO: 37) and oCAB-0297 (SEQ ID NO: 38), the former has a 5′ extension that is complementary to oSGI-JU-0001. The three fragments were assembled, also by PCR using primers oCAB-0294 and pCAB-0297. The purified PCR product was used for transformations.

gRNA was generated using the commercially available MEGAshortscript™ T7 kit, but an RNAse inhibitor was added to the reaction mix. Template was generated by annealing together oligonucleotides oCAB-0341 and oCAB-0342 (SEQ ID Nos: 15-16, respectively).

### Example 5

### Deletion of alg3 Gene

Genome editing for a deletion of a gene can be carried out by transforming the host strain expressing Cas9 with a gRNA targeting a specific site in the genome and a disruption cassette generated using homology arms flanking this site. Homology arms are designed to delete several hundred bases from the genomic sequence.

Deletion of alg3 in the natalizumab Cas9 clone #6920 was carried out by transforming this strain with a linear alg3::nat disruption cassette and gRNA. Nourseothricin resistant colonies were screened for the deletion of alg3 by quantitative PCR (qPCR) using primers oCAB-0298 & oCAB-0299 (SEQ ID Nos: 39-40, respectively). The resulting alg3 deleted strain was given the strain ID #7087.

### Example 6

### Construction of alg3::nat to alg3::bsr Marker Change

Genome editing techniques were utilized to replace the nat marker located in the alg3 gene with a bsr marker. Transformants were screened for the gain of Blasticidin resistance and the loss of Nourseothricin resistance. The presence of previous genome edits was confirmed via resistance to the linked antibiotic resistance markers (paromomycin for the natalizumab heavy chain, hygromycin for the natalizumab light chain, and Zeocin™ for Cas9). Production of natalizumab was confirmed by western blot analysis. The resulting strain was #0394)].

### Example 7

### Construction of Strain #9156 with nat Marker Removed and ku70 Deleted with bsr

Genome editing techniques were utilized to delete ku70 (SG4EUKT582572, SG4EUKT582583, and SG4EUKT561347) in strain #7087. Strain #7087 was transformed with a single guide RNA (gRNA) sequence targeting ku70 and a disruption cassette (ku70::bsr) comprised of a selectable marker (bsr) providing resistance to Blasticidin that is flanked by 5′ and 3′ ku70 homology arms as well as with a single gRNA sequence targeting nat and a disruption cassette (alg3A) comprised of the 5′ and 3′ alg3 homology arms. The deletion of ku70 reduces non-homologous end joining and therefore biases DNA repair by homologous recombination. Transformants were screened initially for the loss of resistance to Nourseothricin (nat), indicating the loss of the nat marker. Clones that were sensitive to Nourseothricin was then screened for the deletion of ku70 by qPCR using primers targeting the deleted region. Production of natalizumab was confirmed by western blot analysis. The resulting strain was given the strain #9156.

### Example 8

### Deletion of Sulfotransferases

Sixteen sulfotransferases were identified as potential sulfotransferases advantageously targeted for deletion or genetic modification in the invention. Sulfotransferases targeted were numbered as 1-16 and SEQ ID Nos: 17-32.

Genome editing experiments were carried out to delete each of the potential sulfotransferases (SEQ ID Nos: 17-32) using disruption cassettes and gRNA for each target sulfotransferase gene in the alg3 deletion strains expressing natalizumab (#0394 or #9156). Organisms were transformed with a single guide RNA (gRNA) sequence targeting each sulfotransferase and a disruption cassette comprised of a selectable marker (nat) providing resistance to Nourseothricin that is flanked by 5′ and 3′ homology arms to target the respective sulfotransferase. Transformants were screened by qPCR using primers targeting the deleted region. Production of natalizumab was confirmed by ELISA. Successful deletions were obtained for SFTs 1-4, 6-7, and 11-16 (SEQ ID Nos: 17-20, 22-23, and 27-32, respectively).

Natalizumab was produced using organisms having deletions of the different sulfotransferases. The final product was purified using protein A and analyzed with a released N-linked glycan method to determine the degree of sulfation.

### Glycan Determination

Various methods are available for determining the percentage of sulfated vs unsulfated glycans in a protein or peptide. Released N-linked glycan can be determined by utilizing an NHS carbamate rapid tagging group, an efficient quinoline fluorophore, and a highly basic tertiary amine for enhancing mass spec ionization. The NHS carbamate hydrolyzes to generate carbon dioxide and a corresponding amine. Convenient commercial kits are available for carrying out the protocol, such as the GlycoWorks® RapiFluor-MS® N-Glycan kit available from Waters® Corporation.

The general procedure for determination of glycans utilized steps of protein denaturation with an anionic surfactant (RapiGest® SF), enzymatic protein deglycosylation (PNGase F), small molecule labeling of released glycan amino group with a mass spec-sensitive derivatizing reagent utilizing an NHS carbamate tagging group that also possesses a strong fluorophore (e.g. Rapifluor-MS®), solid phase extraction-based labeled glycan clean-up to remove excess reagents and contaminant molecules, derivatized glycan separation via hydrophilic interaction liquid chromatography (HILIC) and ultra high performance liquid chromatography (UHPLC), and glycan identification by interpretation of MS data and quantification of glycan abundance by integration of fluorescence signal.

N-glycans were purified chromatographically using an Agilent® 1290 UHPLC system and HILIC chromatography coupled to an LC/MS system using quadrupole time-of-flight technology (i.e. an Agilent® 6520 QT of mass spectrometer) and detected with a fluorescence detector (i.e. an Agilent® 1260 infinity II fluorescence detector). De novo glycan identification was accomplished via interpretation of QT of accurate mass data Once conclusively identified based on MS data, peaks corresponding to the molecules of interest were quantified based on manual integration of their respective fluorescence signals.

This analysis chromatographically resolved the sulfated vs non-sulfated forms of Man3GlcNAc2, Man4GlcNAc2 and Man5GlcNAc2, which accounted for more than 80% of the glycans present on the natalizumab antibodies produced in organism #0394. The total amount of sulfated Man(3-5)GlcNAc2(ManS) and the total amount of non-sulfated Man(3-5)GlcNAc2(Man) expressed individually as a percentage of all glycans observed are presented in graphical form in FIG. 1. Organism #0394 without sulfotransferase deletion generated an antibody with 69% a ManS and 29% Man while deletion of SFT15 (SEQ ID NO: 31) significantly decreased ManS (48%) and increased Man (45%). Significant decreases of ManS and increases in Man were also observed with deletions of SFT12 (SEQ ID NO: 28) and SFT16 (SEQ ID NO: 32).

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description, and there is no intention in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described as if set forth individually herein. Sub-headings are used for organizational purposes only and to assist the reader, and should not be construed as limiting the disclosure. Other embodiments are within the following claims.

Although the invention has been described with reference to the above examples, it will be understood that modifications and variations are encompassed within the spirit and scope of the invention. Accordingly, the invention is limited only by the following claims.

1. A recombinant cell of the Family Thraustochytriaceae comprising a nucleic acid encoding a heterologous glycomolecule, and further comprising a genetic modification in one or more sulfotransferase gene(s), wherein the one or more sulfotransferase genes comprise a sequence having at least 80% sequence identity to a sequence selected from the group consisting of: SEQ ID NO: 28, 31, and 32.

2. The recombinant cell of claim 1 wherein the genetic modification is selected from the group consisting of: a deletion, a mutation, a disruption, an insertion, an inactivation, an attenuation, and an inversion.

3. (canceled)

4. The recombinant cell of claim 1 wherein the glycomolecule is a glycoprotein or glycopeptide.

5-9. (canceled)

10. The recombinant cell of claim 1 wherein the cell produces and secretes the heterologous polypeptide molecule or functional portion thereof.

11. The recombinant cell of claim 1 wherein the glycomolecule is an immunoglobulin.

12. The recombinant cell of claim 11 wherein the heterologous immunoglobulin comprises fewer sulfated N-glycans relative to a corresponding cell that does not comprise the genetic modification.

13. The recombinant cell of claim 12 wherein the heterologous immunoglobulin comprises at least 30% unsulfated N-glycans relative to a corresponding cell that does not comprise the genetic modification.

14. The recombinant cell of claim 11 wherein the heterologous immunoglobulin comprises at least 40% unsulfated N-glycans relative to a corresponding cell that does not comprise the genetic modification.

15. The recombinant cell of claim 12 wherein the ratio of unsulfated to sulfated N-glycans in the heterologous immunoglobulin is at least 1:2.

16. The recombinant cell of claim 12 wherein the N-glycans comprise Man₃₋₅GlcNAc₂.

17. The recombinant cell of claim 1 wherein the glycomolecule is an antibody molecule, or portion thereof.

18. (canceled)

19. The recombinant cell of claim 1 wherein the Thraustochytriaceae cell is of a genus selected from the group consisting of: Japanochytrium, Oblongichytrium, Thraustochytrium, Aurantiochytrium, and Schizochytrium.

20. (canceled)

21. The recombinant cell of claim 1 further comprising a genetic modification to a gene encoding a mannosyl transferase.

22. The recombinant cell of claim 21 wherein the genetic modification is comprised in an alg3 gene.

23. The recombinant cell of claim 22 wherein the genetic modification is selected from the group consisting of: a deletion, a mutation, a disruption, an insertion, an inactivation, replacement, an attenuation, and an inversion.

24. The recombinant cell of claim 1 wherein the heterologous glycoprotein or glycopeptide is selected from the group consisting of: trastuzumab, eculizumab, natalizumab, cetuximab, omalizumab, usteinumab, panitummab, and adalimumab, or a functional fragment of any of them.

25.-26. (canceled)

27. A method of producing a glycomolecule having a glycosylation profile with low sulfation, comprising
performing a genetic modification to one or more sulfotransferase genes in an organism of the Family Thraustochytriales, wherein the organism comprises a recombinant nucleic acid encoding a heterologous glycomolecule, or a functional portion thereof and wherein the one or more sulfotransferase gene(s) comprises a sequence having at least 80% sequence identity to a sequence selected from the group consisting of: SEQ ID NO: 28, 31, and 32;
cultivating the organism; and
thereby producing the glycomolecule having a glycosylation profile with low sulfation.

28. The method of claim 27 wherein the genetic modification is selected from the group consisting of: a deletion, a mutation, a disruption, an insertion, an inactivation, an attenuation, and an inversion.

29-31. (canceled)

32. The method of claim 27 wherein the organism secretes a heterologous glycoprotein or glycopeptide molecule or functional portion thereof.

33. The method of claim 32 wherein the heterologous glycoprotein or glycopeptide molecule comprises fewer sulfated N-glycans relative to the heterologous glycoprotein or glycopeptide produced in a corresponding cell that does not comprise the genetic modification.

34.-41. (canceled)
